(19) United States
(12) Patent Application Publication
(10) Pub. No.: US 2022/0016060 A1
(43) Pub. Date: Jan. 20, 2022

(54) USE OF SODIUM 2-(3-PENTYLPHENYL)ACETATE IN THE TREATMENT OF ALSTRÖM SYNDROME

(71) Applicant: LIMINAL BIOSCIENCES LIMITED, COMBERTON CAMBRIDGESHIRE (GB)

(72) Inventors: LYNE GAGNON, LAVAL, QUEBEC (CA); PIERRE LAURIN, VILLE MONT-ROYAL, QUEBEC (CA); FRANK CESARI, POINTE-CLAIRE, QUEBEC (CA)

(21) Appl. No.: 17/311,616

(22) PCT Filed: Dec. 5, 2019
(86) PCT No.: PCT/CA2019/051753

§ 371 Date: Jun. 7, 2021

Related U.S. Application Data
(60) Provisional application No. 62/775,849, filed on Dec. 5, 2018.

(51) Int. Cl. A61K 31/192; A61P 1/16; A61P 9/00; A61P 13/12; A61P 3/00; A61P 3/08; A61P 3/10

(52) U.S. Cl. CPC A61K 31/192; A61P 1/16; A61P 9/00; A61P 3/10; A61P 3/00; A61P 3/08; A61P 13/12

(57) ABSTRACT

Methods, formulations/compositions and uses for improving at least one of liver histology, heart histology, kidney histology, adipose tissue histology and a metabolic parameter in a patient suffering from Alström syndrome are described, comprising administration of a pharmaceutical oral formulation comprising 2-(3-pentylphenyl)acetate or a pharmaceutically salt thereof and a pharmaceutically acceptable excipient at a daily dose of about 500 mg to about 1500 mg. The treatment is preferably for a period of more than 48 weeks and is not interrupted for 15 days or more.

### CROSS REFERENCE TO RELATED APPLICATIONS

This application claims the benefit of U.S. provisional application Ser. No. 62/775,849, filed on Dec. 5, 2018, which is incorporated herein by reference in its entirety.

### TECHNICAL FIELD

The present disclosure generally relates to the management of pathological features of Alström syndrome (ALMS).

### BACKGROUND ART

ALMS is a rare autosomal recessive genetic disorder with an estimated prevalence of less than one per million. It is characterized by cone-rod dystrophy, hearing loss, childhood truncal obesity, insulin resistance and hyperinsulinemia, type 2 diabetes, hypertriglyceridemia, short stature in adulthood, cardiomyopathy, and progressive pulmonary, hepatic, and renal dysfunction. Symptoms first appear in infancy and progressive development of multi-organ pathology leads to a reduced life expectancy. Variability in age of onset and severity of clinical symptoms, even within families, is likely due to genetic background. The severity of the disease, often leading to organ failure, results in a reduced life expectancy, rarely exceeding 50 years. The clinical care of individuals is complex due to the combination of multiple endocrine disorders, sensorineural deficits, cardiac, renal, and hepatic abnormalities. There is no specific therapy, and individuals are treated and monitored on the basis of individual symptoms.

There is thus a need for novel therapeutic approaches for the management of ALMS.

The present description refers to a number of documents, the content of which is herein incorporated by reference in their entirety.

### SUMMARY OF THE DISCLOSURE

The present disclosure relates to the management of pathological features of ALMS.

More specifically, in accordance with aspects and embodiments of the present disclosure, there are provided the following items:
1. A method for improving at least one of liver histology, heart histology, kidney histology, adipose tissue histology and glycemic control in a patient suffering from ALMS, said method comprising administering to said patient a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg for a treatment period of more than 48 weeks, and wherein the administration is not interrupted for 15 days or more during said treatment period.
2. The method of item 1, wherein the treatment period is of more than 54 weeks.
3. The method of item 1, wherein the treatment period is of more than 60 weeks.
4. The method of any one of items 1 to 3, wherein the administration is not interrupted for 30 days or more.
5. The method of any one of items 1 to 4, wherein said method is for improving at least liver histology.
6. The method of item 5, wherein improving liver histology comprises reducing liver stiffness.
7. The method of item 6, wherein the liver stiffness is reduced by at least 2 kPa relative to prior to the administration, as measured by transient elastography.
8. The method of item 7, wherein the liver stiffness is reduced by at least 2.25 kPa relative to prior to the administration, as measured by transient elastography.
9. The method of any one of items 5 to 8, wherein improving liver histology comprises reducing the Metavir score by at least one grade or stage.
10. The method of any one of items 1 to 9, wherein said method is for improving at least heart histology.
11. The method of any one of items 1 to 10, wherein improving heart histology comprises increasing left ventricular function (LVF).
12. The method of item 11, wherein the LVF comprises left ventricular end-diastolic volume (LVEDV) and/or left ventricular end-systolic volume (LVESV).
13. The method of item 11, wherein the LVF comprises LVEDV.
14. The method of item 12 or 13, wherein the method is for increasing LVEDV by at least 6 ml relative to prior to the administration, as measured by cardiac Magnetic Resonance Imaging (MRI).
15. The method of any one of items 10 to 14, wherein improving heart histology comprises reducing short-axis (plane) longitudinal relaxation time (SAX T1).
16. The method of item 15, wherein the SAX T1 is Basal SAX T1 and/or Mid SAX T1.
17. The method of item 16, wherein the method is for reducing Basal SAX T1 by at least 40 ms relative to prior to the administration, as measured by cardiac MRI.
18. The method of item 16 or 17, wherein the method is for reducing Mid SAX T1 by at least 25 ms relative to prior to the administration, as measured by cardiac MRI.
19. The method of any one of items 1 to 18, wherein said method is for improving at least kidney histology.
20. The method of any one of items 1 to 19, wherein improving kidney histology comprises restricting kidney damage or injury.
21. The method of item 19 or 20, wherein improving kidney histology comprises reducing the levels of one or more biomarkers of kidney injury relative to prior to the administration.
22. The method of item 21, wherein the one or more biomarkers of kidney injury are monocyte chemoattractant protein 1 (MCP-1), kidney injury molecule-1 (KIM-1), clusterin, cystatin C and osteopontin.
23. The method of item 22, wherein the method is for reducing the levels of said one or more biomarkers by at least 10%.
24. The method of any one of items 1 to 23, wherein said method is for improving at least adipose tissue histology.
25. The method of item 24, wherein improving adipose tissue histology comprises reducing average adipocyte area.
26. The method of item 25, wherein the method is for reducing average adipocyte area by at least 10%.
27. The method of any one of items 1 to 26, wherein said method is for improving at least glycemic control.
28. The method of any one of items 1 to 27, wherein improving glycemic control comprises reducing glycated hemoglobin (HbA1c).
29. The method of item 28, wherein the subject has an HbA1c of more than 9% prior to administration.
30. The method of item 28 or 29, wherein the method is for reducing HbA1c by at least 1% (absolute).
31. The method of any one of items 1 to 30, wherein improving glycemic control comprises increasing the homeostasis Model Assessment for Steady State Beta-Cell Function Based on C-Peptide and Fasting Plasma Glucose (HOMA-B [C-Peptide/FPG]).
32. The method of item 31, wherein said method is for increasing HOMA-B [C-Peptide/FPG] by at least 20.
33. A method for improving glycemic control in an ALMS patient with diabetes, said method comprising administering to said patient a pharmaceutical oral formulation comprising 2-(3-pentylphenyl)acetate or a pharmaceutically salt thereof and a pharmaceutically acceptable excipient, wherein said 2-(3-pentylphenyl)acetate or pharmaceutically salt thereof is administered at a daily dose of about 500 mg to about 1500 mg.
34. The method of item 33, wherein the patient has type 2 diabetes.
35. A method for improving liver function in an ALMS patient with liver disease, said method comprising administering to said patient a pharmaceutical oral formulation comprising 2-(3-pentylphenyl)acetate or a pharmaceutically salt thereof and a pharmaceutically acceptable excipient, wherein said 2-(3-pentylphenyl)acetate or pharmaceutically salt thereof is administered at a daily dose of about 500 mg to about 1500 mg.
36. The method of item 35, wherein the liver disease comprises liver fibrosis.
37. The method of item 36, wherein the patient has fibrosis stage F2, F3 or F4 according to the Metavir scoring system.
38. A method for improving cardiac function in an ALMS patient, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.
39. The method of item 38, wherein the cardiac function is left ventricular function (LVF) and/or short-axis (plane) longitudinal relaxation time (SAX T1).
40. The method of item 39, wherein the LVF comprises left ventricular end-diastolic volume (LVEDV) and/or left ventricular end-systolic volume (LVESV).
41. The method of item 39 or 40, wherein the patient has a lower than normal LVF.
42. The method of item 41, wherein (i) the patient is a male and has a LVEDV of less than 106 mL and/or a LVESV of less than 26 mL; or (ii) the patient is a female and has a LVEDV of less than 86 mL and/or a LVESV of less than 22 mL.
43. A method for improving kidney function in an ALMS patient suffering from kidney damage or injury, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.
44. The method of item 43, wherein the patient has higher than normal levels of one or more biomarkers of kidney toxicity.
45. The method of item 44, wherein the one or more biomarkers of kidney injury are monocyte chemoattractant protein 1 (MCP-1), kidney injury molecule-1 (KIM-1), clusterin, cystatin C and osteopontin.
46. A method for improving adipose tissue function in an ALMS patient, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.
47. The method of item 46, wherein the adipose tissue function is perivascular adipose tissue function.
48. The method of any one of items 33 to 47, wherein said 2-(3-pentylphenyl)acetate or a pharmaceutically salt thereof is administered for a treatment period of at least 24 weeks.
49. The method of item 48, wherein said 2-(3-pentylphenyl)acetate or a pharmaceutically salt thereof is administered for a treatment period of at least 48 weeks.
50. The method of item 48, wherein said 2-(3-pentylphenyl)acetate or a pharmaceutically salt thereof is administered for a treatment period of at least 60 weeks.
51. The method of any one of items 1 to 50, wherein the daily dose is of about 700 mg to about 900 mg.
52. The method of item 51, wherein the daily dose is of about 800 mg.
53. The method of any one of items 1 to 50, wherein the daily dose is of about 1100 mg to about 1300 mg.
54. The method of item 53, wherein the daily dose is of about 1200 mg.
55. The method of any one of items 1 to 54, wherein the pharmaceutical oral formulation comprises sodium 2-(3-pentylphenyl)acetate.
56. The method of any one of items 1 to 55, wherein the pharmaceutical oral formulation is a capsule or a tablet.
57. The method of any one of items 1 to 56, wherein the subject has a body mass index (BMI) 25.0 kg/m².
58. The method of any one of items 1 to 57, wherein the subject is a male and has a waist circumference 94 cm, or the subject is a female and has a waist circumference 80 cm.
59. A pharmaceutical oral formulation for use in improving at least one of liver histology, heart histology, kidney histology, adipose tissue histology and glycemic control in a patient suffering from ALMS, the pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is for administration at a daily dose of about 500 mg to about 1500 mg for a treatment period of more than 48 weeks, and wherein the administration is not interrupted for 15 days or more during said treatment period.
60. The formulation for use of item 59, wherein the treatment period is of more than 54 weeks.
61. The formulation for use of item 59, wherein the treatment period is of more than 60 weeks.
62. The formulation for use of any one of items 59 to 61, wherein the administration is not interrupted for 30 days or more.
63. The formulation for use of any one of items 59 to 62, wherein said method is for improving at least liver histology.
64. The formulation for use of item 63, wherein improving liver histology comprises reducing liver stiffness.
65. The formulation for use of item 64, wherein the liver stiffness is reduced by at least 2 kPa relative to prior to the administration, as measured by transient elastography.
66. The formulation for use of item 65, wherein the liver stiffness is reduced by at least 2.25 kPa relative to prior to the administration, as measured by transient elastography.
67. The formulation for use of any one of items 63 to 68, wherein improving liver histology comprises reducing the Metavir score by at least one grade or stage.
68. The formulation for use of any one of items 59 to 67, wherein said method is for improving at least heart histology.
69. The formulation for use of any one of items 59 to 70, wherein improving heart histology comprises increasing left ventricular function (LVF)
70. The formulation for use of item 69, wherein the LVF comprises left ventricular end-diastolic volume (LVEDV) and/or left ventricular end-systolic volume (LVESV).
71. The formulation for use of item 70, wherein the LVF comprises LVEDV.
72. The formulation for use of item 70 or 71, wherein the method is for increasing LVEDV by at least 6 ml relative to prior to the administration, as measured by cardiac Magnetic Resonance Imaging (MRI).
73. The formulation for use of any one of items 68 to 71, wherein improving heart histology comprises reducing short-axis (plane) longitudinal relaxation time (SAX T1).
74. The formulation for use of item 773, wherein the SAX T1 is Basal SAX T1 and/or Mid SAX T1.
75. The formulation for use of item 74, wherein the method is for reducing Basal SAX T1 by at least 40 ms relative to prior to the administration, as measured by cardiac MRI.
76. The formulation for use of item 74 or 75, wherein the method is for reducing Mid SAX T1 by at least 25 ms relative to prior to the administration, as measured by cardiac MRI.
77. The formulation for use of any one of items 59 to 76, wherein said method is for improving at least kidney histology.
78. The formulation for use of any one of items 59 to 77, wherein improving kidney histology comprises restricting kidney damage or injury.
79. The formulation for use of item 77 or 78, wherein improving kidney histology comprises reducing the levels of one or more biomarkers of kidney injury relative to prior to the administration.
80. The formulation for use of item 79, wherein the one or more biomarkers of kidney injury are monocyte chemoattractant protein 1 (MCP-1), kidney injury molecule-1 (KIM-1), clusterin, cystatin C and osteopontin.
81. The formulation for use of item 80, wherein the method is for reducing the levels of said one or more biomarkers by at least 10%.
82. The formulation for use of any one of items 59 to 81, wherein said method is for improving at least adipose tissue histology.
83. The formulation for use of item 82, wherein improving adipose tissue histology comprises reducing average adipocyte area.
84. The formulation for use of item 83, wherein the method is for reducing average adipocyte area by at least 10%.
85. The formulation for use of any one of items 59 to 84, wherein said method is for improving at least glycemic control.
86. The formulation for use of any one of items 1 to 85, wherein improving glycemic control comprises reducing glycated hemoglobin (HbA1c).
87. The formulation for use of item 86, wherein the subject has an HbA1c of more than 9% prior to administration.
88. The formulation for use of item 86 or 87, wherein the method is for reducing HbA1c by at least 1% (absolute).
89. The formulation for use of any one of items 59 to 88, wherein improving glycemic control comprises increasing the homeostasis Model Assessment for Steady State Beta-Cell Function Based on C-Peptide and Fasting Plasma Glucose (HOMA-B [C-Peptide/FPG]).
90. The formulation for use of item 89, wherein said method is for increasing HOMA-B [C-Peptide/FPG] by at least 20.
91. A pharmaceutical oral formulation for use in improving glycemic control in an ALMS patient with diabetes, said pharmaceutical oral formulation comprising 2-(3-pentylphenyl)acetate or a pharmaceutically salt thereof and a pharmaceutically acceptable excipient, wherein said 2-(3-pentylphenyl)acetate or pharmaceutically salt thereof is administered at a daily dose of about 500 mg to about 1500 mg.
92. The formulation for use of item 91, wherein the patient has type 2 diabetes.
93. A pharmaceutical oral formulation for use in improving liver function in an ALMS patient with liver disease, said pharmaceutical oral formulation comprising 2-(3-pentylphenyl)acetate or a pharmaceutically salt thereof and a pharmaceutically acceptable excipient, wherein said 2-(3-pentylphenyl)acetate or pharmaceutically salt thereof is administered at a daily dose of about 500 mg to about 1500 mg.
94. The formulation for use of item 93, wherein the liver disease comprises liver fibrosis.
95. The formulation for use of item 94, wherein the patient has fibrosis stage F2, F3 or F4 according to the Metavir scoring system.
96. A pharmaceutical oral formulation for use in improving cardiac function in an ALMS patient, said pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.
97. The formulation for use of item 96, wherein the cardiac function is left ventricular function (LVF) and/or short-axis (plane) longitudinal relaxation time (SAX T1).
98. The formulation for use of item 97, wherein the LVF comprises left ventricular end-diastolic volume (LVEDV) and/or left ventricular end-systolic volume (LVESV).
99. The formulation for use of item 97 or 98, wherein the patient has a lower than normal LVF.
100. The formulation for use of item 99, wherein (i) the patient is a male and has a LVEDV of less than 106 mL and/or a LVESV of less than 26 mL; or (ii) the patient is a female and has a LVEDV of less than 86 mL and/or a LVESV of less than 22 mL.
101. A pharmaceutical oral formulation for use in improving kidney function in an ALMS patient suffering from kidney damage or injury, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.
102. The formulation for use of item 101, wherein the patient has higher than normal levels of one or more biomarkers of kidney toxicity.
103. The formulation for use of item 102, wherein the one or more biomarkers of kidney injury are monocyte chemoattractant protein 1 (MCP-1), kidney injury molecule-1 (KIM-1), clusterin, cystatin C and osteopontin.
104. A pharmaceutical oral formulation for use in improving adipose tissue function in an ALMS patient, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.
105. The formulation for use of item 104, wherein the adipose tissue function is perivascular adipose tissue function.
106. The formulation for use of any one of items 91 to 105, wherein said 2-(3-pentylphenyl)acetate or a pharmaceutically salt thereof is administered for a treatment period of at least 24 weeks.
107. The formulation for use of item 106, wherein said 2-(3-pentylphenyl)acetate or a pharmaceutically salt thereof is administered for a treatment period of at least 48 weeks.
108. The formulation for use of item 106, wherein said 2-(3-pentylphenyl)acetate or a pharmaceutically salt thereof is administered for a treatment period of at least 60 weeks.
109. The formulation for use of any one of items 59 to 108, wherein the daily dose is of about 700 mg to about 900 mg.
110. The formulation for use of item 109, wherein the daily dose is of about 800 mg.
111. The formulation for use of any one of items 59 to 108, wherein the daily dose is of about 1100 mg to about 1300 mg.
112. The formulation for use of item 111, wherein the daily dose is of about 1200 mg.
113. The formulation for use of any one of items 59 to 112, wherein the pharmaceutical oral formulation comprises sodium 2-(3-pentylphenyl)acetate.
114. The formulation for use of any one of items 59 to 113, wherein the pharmaceutical oral formulation is a capsule or a tablet.
115. The formulation for use of any one of items 59 to 114, wherein the subject has a body mass index (BMI) 25.0 kg/m².
116. The formulation for use of any one of items 59 to 115, wherein the subject is a male and has a waist circumference 94 cm, or the subject is a female and has a waist circumference 80 cm.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts a flowchart of study design.

FIGS. 2A and 2B are histological images of adipocyte tissue in an ALMS subject before (FIG. 2A) and after (FIG. 2B) 24 weeks of treatment with sodium 2-(3-pentylphenyl)acetate. In FIG. 2A, asterisks (*) represent coalescence of adipocytes forming bigger vesicular vacuolation/steatosis (macrovesicular vacuolation).

FIGS. 3A and 3B are histological images of adipocyte tissue artery in an ALMS subject before (FIG. 3A) and after (FIG. 3B) 24 weeks of treatment with sodium 2-(3-pentylphenyl)acetate.

### DESCRIPTION OF ILLUSTRATIVE EMBODIMENTS

The use of the terms “a” and “an” and “the” and similar referents in the context of describing the various aspects and embodiments disclosed herein (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Any and all combinations and subcombinations of the embodiments and features disclosed herein are encompassed by the present disclosure.

All methods described herein can be performed in any suitable order unless otherwise indicated herein or otherwise clearly contradicted by context.

The use of any and all examples, or exemplary language (e.g., “such as”) provided herein, is intended merely to better illustrate the disclosure and does not pose a limitation on the scope of the disclosure unless otherwise claimed.

No language in the specification should be construed as indicating any non-claimed element as essential to the practice of the various aspects and embodiments disclosed herein.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

In the study described herein, sustained administration of 800 mg of sodium 2-(3-pentylphenyl)acetate improves the pathological features of several organs/tissues, as well as certain metabolic parameters, in subjects suffering from ALMS, and that significant interruption of treatment within the treatment period may lead to worsening of symptoms.

Accordingly, in a first aspect, the present disclosure provides a method for improving at least one of liver histology, heart histology, kidney histology adipocyte histology and a metabolic parameter (e.g., glycemic control) in a subject suffering from ALMS, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or pharmaceutically salt thereof is administered at a daily dose of about 500 mg to about 1500 mg for a treatment period of more than 48 weeks, and wherein the administration is not interrupted for 15 days or more during said treatment period.

The present disclosure also provides the use of a pharmaceutical oral formulation comprising 2-(3-pentylphenyl)acetate or a pharmaceutically salt thereof and a pharmaceutically acceptable excipient for improving at least one of liver histology, heart histology, kidney histology adipocyte histology and a metabolic parameter (e.g., glycemic control) in a subject suffering from ALMS, wherein said 2-(3-pentylphenyl)acetate or pharmaceutically salt thereof is for administration at a daily dose of about 500 mg to about 1500 mg for a treatment period of more than 48 weeks, and wherein there is no treatment interruption of 15 days or more during said treatment period.

The present disclosure also provides the use of a pharmaceutical oral formulation comprising 2-(3-pentylphenyl)acetate or a pharmaceutically salt thereof and a pharmaceutically acceptable excipient for the manufacture of a medicament for improving at least one of liver histology, heart histology, kidney histology adipocyte histology and a metabolic parameter (e.g., glycemic control) in a subject suffering from ALMS, wherein said medicament is for administration of 2-(3-pentylphenyl)acetate or pharmaceutically salt thereof at a daily dose of about 500 mg to about 1500 mg for a treatment period of more than 48 weeks, and wherein there is no treatment interruption of 15 days or more during said treatment period.

The present disclosure also provides a pharmaceutical oral formulation comprising 2-(3-pentylphenyl)acetate or a pharmaceutically salt thereof and a pharmaceutically acceptable excipient for use in improving at least one of liver histology, heart histology, kidney histology, adipose tissue histology and a metabolic parameter (e.g., glycemic control) in a subject suffering from ALMS, wherein said 2-(3-pentylphenyl)acetate or pharmaceutically salt thereof is for administration at a daily dose of about 500 mg to about 1500 mg for a treatment period of more than 48 weeks, and wherein there is no treatment interruption of 15 days or more during said treatment period.

The present disclosure also provides a method for improving glycemic control in an ALMS patient with diabetes, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or pharmaceutically salt thereof is administered at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for improving glycemic control in an ALMS patient with diabetes, wherein said 2-(3-pentylphenyl)acetate or pharmaceutically salt thereof is for administration at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for the manufacture of a medicament for improving glycemic control in an ALMS patient with diabetes, wherein said medicament is for administration of 2-(3-pentylphenyl)acetate or pharmaceutically salt thereof at a daily dose of about 500 mg to about 1500 mg.

The present disclosure also provides a pharmaceutical oral formulation comprising 2-(3-pentylphenyl)acetate or a pharmaceutically salt thereof and a pharmaceutically acceptable excipient for use in improving glycemic control in an ALMS patient with diabetes, wherein said 2-(3-pentylphenyl)acetate or pharmaceutically salt thereof is for administration at a daily dose of about 500 mg to about 1500 mg.

The present disclosure also provides a method for improving liver function in an ALMS patient with liver disease, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or pharmaceutically salt thereof is administered at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for improving liver function in an ALMS patient with liver fibrosis (e.g., with stage F2, F3 or F4 fibrosis according to the Metavir scoring system), wherein said 2-(3-pentylphenyl)acetate or pharmaceutically salt thereof is for administration at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for the manufacture of a medicament for improving liver function in an ALMS patient with liver fibrosis (e.g., with stage F2, F3 or F4 fibrosis according to the Metavir scoring system), wherein said medicament is for administration of 2-(3-pentylphenyl)acetate or pharmaceutically salt thereof at a daily dose of about 500 mg to about 1500 mg.

The present disclosure also provides a pharmaceutical oral formulation comprising 2-(3-pentylphenyl)acetate or a pharmaceutically salt thereof and a pharmaceutically acceptable excipient for use in improving liver function in an ALMS patient with liver fibrosis (e.g., with stage F2, F3 or F4 fibrosis according to the Metavir scoring system), wherein said 2-(3-pentylphenyl)acetate or pharmaceutically salt thereof is for administration at a daily dose of about 500 mg to about 1500 mg.

In an embodiment, the liver disease comprises liver fibrosis, e.g., stage F2, F3 or F4 fibrosis according to the Metavir scoring system.

The present disclosure also provides a method for improving left ventricular function in an ALMS patient, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for improving left ventricular function in an ALMS patient, wherein said 2-(3-pentylphenyl)acetate or pharmaceutically salt thereof is for administration at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for the manufacture of a medicament for improving left ventricular function in an ALMS patient, wherein said medicament is for administration of 2-(3-pentylphenyl)acetate or pharmaceutically salt thereof at a daily dose of about 500 mg to about 1500 mg.

The present disclosure also provides a pharmaceutical oral formulation comprising 2-(3-pentylphenyl)acetate or a pharmaceutically salt thereof and a pharmaceutically acceptable excipient for use in improving left ventricular function in an ALMS patient, wherein said 2-(3-pentylphenyl)acetate or pharmaceutically salt thereof is for administration at a daily dose of about 500 mg to about 1500 mg.

In an embodiment, the LVF comprises left ventricular end-diastolic volume (LVEDV) and/or left ventricular end-systolic volume (LVESV). In an embodiment, the ALMS patient has a left ventricular function that is lower than the normal range. The normal LVEDV range is 106-214 mL for males and 86-178 mL for females, and the normal LVESV range is 26-82 mL for males and 22-66 mL for females. In an embodiment, the patient is a male and has a LVEDV of less than 106 mL and/or a LVESV of less than 26 mL. In another embodiment, the patient is a female and has a LVEDV of less than 86 mL and/or a LVESV of less than 22 mL.

The present disclosure also provides a method for improving short-axis (plane) longitudinal relaxation time (SAX T1) in an ALMS patient, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for improving short-axis (plane) longitudinal relaxation time (SAX T1) in an ALMS patient, wherein said 2-(3-pentylphenyl)acetate or pharmaceutically salt thereof is for administration at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for the manufacture of a medicament for improving short-axis (plane) longitudinal relaxation time (SAX T1) in an ALMS patient, wherein said medicament is for administration of 2-(3-pentylphenyl)acetate or pharmaceutically salt thereof at a daily dose of about 500 mg to about 1500 mg.

The present disclosure also provides a pharmaceutical oral formulation comprising 2-(3-pentylphenyl)acetate or a pharmaceutically salt thereof and a pharmaceutically acceptable excipient for use in improving short-axis (plane) longitudinal relaxation time (SAX T1) in an ALMS patient, wherein said 2-(3-pentylphenyl)acetate or pharmaceutically salt thereof is for administration at a daily dose of about 500 mg to about 1500 mg.

The present disclosure also provides a method for improving kidney function in an ALMS patient suffering from kidney damage or injury, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for improving kidney function in an ALMS patient suffering from kidney damage or injury, wherein said 2-(3-pentylphenyl)acetate or pharmaceutically salt thereof is for administration at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for the manufacture of a medicament for improving kidney function in an ALMS patient suffering from kidney damage or injury, wherein said medicament is for administration of 2-(3-pentylphenyl)acetate or pharmaceutically salt thereof at a daily dose of about 500 mg to about 1500 mg.

The present disclosure also provides a pharmaceutical oral formulation comprising 2-(3-pentylphenyl)acetate or a pharmaceutically salt thereof and a pharmaceutically acceptable excipient for use in improving kidney function in an ALMS patient suffering from kidney damage or injury, wherein said 2-(3-pentylphenyl)acetate or pharmaceutically salt thereof is for administration at a daily dose of about 500 mg to about 1500 mg.

The present disclosure also provides a method for improving adipose tissue function (e.g., perivascular adipose tissue function) in an ALMS patient, said method comprising administering to said subject a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for improving adipose tissue function (e.g., perivascular adipose tissue function) in an ALMS patient, wherein said 2-(3-pentylphenyl)acetate or pharmaceutically salt thereof is for administration at a daily dose of about 500 mg to about 1500 mg.

The present disclosure also provides the use of a pharmaceutical oral formulation comprising 2-(3-pentylphenyl)acetate or a pharmaceutically salt thereof and a pharmaceutically acceptable excipient for the manufacture of a medicament for improving adipose tissue function (e.g., perivascular adipose tissue function) in an ALMS patient, wherein said medicament is for administration of 2-(3-pentylphenyl)acetate or pharmaceutically salt thereof at a daily dose of about 500 mg to about 1500 mg.

The present disclosure also provides a pharmaceutical oral formulation comprising 2-(3-pentylphenyl)acetate or a pharmaceutically salt thereof and a pharmaceutically acceptable excipient for use in improving adipose tissue function (e.g., perivascular adipose tissue function) in an ALMS patient, wherein said 2-(3-pentylphenyl)acetate or pharmaceutically salt thereof is for administration at a daily dose of about 500 mg to about 1500 mg.

2-(3-pentylphenyl)acetate has the following structure (shown as the corresponding acid):

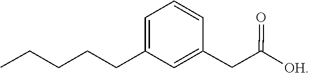

The salt of 2-(3-pentylphenyl)acetate is a pharmaceutically acceptable salt. The term “pharmaceutically acceptable salt” refers to salts of 2-(3-pentylphenyl)acetate that are pharmacologically acceptable and substantially non-toxic to the subject to which they are administered. More specifically, these salts retain the biological effectiveness and properties of 2-(3-pentylphenyl)acetate and are formed from suitable non-toxic organic or inorganic acids or bases.

In an embodiment, the salt is a base salt formed with an inorganic or organic base. Such salts include alkali metal salts such as sodium, lithium, and potassium salts; alkaline earth metal salts such as calcium and magnesium salts; metal salts such as aluminum salts, iron salts, zinc salts, copper salts, nickel salts and a cobalt salts; inorganic amine salts such as ammonium or substituted ammonium salts, such as trimethylammonium salts; and salts with organic bases (for example, organic amines) such as chloroprocaine salts, dibenzylamine salts, dicyclohexylamine salts, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amines salts, tetramethylammonium salts, t-octylamine salts, tris-(2-hydroxyethyl)amine salts, and tris(hydroxymethyl)aminomethane salts. In an embodiment, the pharmaceutically acceptable base salt of 2-(3-pentylphenyl)acetate is a metal salt, preferably a sodium salt. Thus, in an embodiment, the pharmaceutical oral formulation comprises sodium 2-(3-pentylphenyl)acetate, also referred to as 3-pentylbenzeneacetic acid sodium salt, which has the following structure:

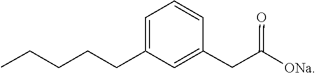

In an embodiment, the method, formulation or use is for at least improving liver histology. In an embodiment, the method, formulation or use is for at least improving heart histology. In an embodiment, the method, formulation or use is for at least improving kidney histology. In an embodiment, the method, formulation or use is for at least improving adipose tissue histology. In an embodiment, the method, formulation or use is for at least improving a metabolic parameter (e.g., glycemic control). In an embodiment, the method, formulation or use is for improving at least two features among liver histology, heart histology, kidney histology, adipose tissue histology (e.g., perivascular adipose tissue histology) and a metabolic parameter (e.g., glycemic control). In an embodiment, the method, formulation or use is for improving at least three features among liver histology, heart histology, kidney histology, adipose tissue histology and a metabolic parameter (e.g., glycemic control). In an embodiment, the method, formulation or use is for improving at least four features among liver histology, heart histology, kidney histology, adipose tissue histology and a metabolic parameter (e.g., glycemic control). In an embodiment, the method, formulation or use is for improving liver histology, heart histology, kidney histology, adipose tissue histology and a metabolic parameter (e.g., glycemic control).

The terms “improving liver histology”, “improving heart histology”, “improving kidney histology”, and “improving adipose tissue histology” as used herein means that the method, formulation or use corrects or slows the progression of one or more pathological features associated with ALMS that was present in the recited organ or tissue from the patient prior to the treatment. The improvement may be determined by comparing the extent or severity of the one or more pathological features prior to and after the treatment period using suitable tests or assays. Improvement may also mean that the extent or severity of the one or more histopathological features has not worsened (i.e. is stable) after the treatment period or has worsened at a slower pace after the treatment period relative to an untreated patient (i.e. the progression of the pathological feature is slowed by the treatment).

Pathological features associated with ALMS include alterations of the normal structure or architecture of the organ or tissue that generally interfere with normal organ or tissue function, such as the presence of damages or injuries in the organ or tissue, the presence of scar tissue, and/or the presence of abnormal cells in the organ or tissue.

One of the pathological features associated with ALMS in the liver is increased liver stiffness. Thus, in embodiments, improving liver histology comprises reducing liver stiffness, or slowing the progression of liver stiffness. Liver stiffness may be determined using well-known methods and assays, including transient elastography (e.g., using FibroScan®). In an embodiment, the liver stiffness is reduced by at least 2 kPa relative to prior to the administration, as measured by transient elastography. In another embodiment, the liver stiffness is reduced by at least 2.1, 2.2, 2.25, 2.3, 2.35, 2.4, 2.45 or 2.5 kPa relative to prior to the administration, as measured by transient elastography. The extent or severity of liver stiffness may also be assessed using suitable scoring systems, such as the Metavir scoring system. The METAVIR scoring system is used to assess the extent of inflammation and fibrosis by histopathological evaluation in a liver biopsy of patients, and includes the following grades and stages:

Activity grade: A0=no activity; A1=mild activity; A2: moderate activity; A3: severe activity

Fibrosis stage: F0=no fibrosis; F1=portal fibrosis without septa; F2=portal fibrosis with few septa; F3=numerous septa without cirrhosis; F4=cirrhosis.

In an embodiment, improving liver histology comprises reducing the Metavir score by at least one grade or stage, e.g., reduction in liver fibrosis by at least one grade. In another embodiment, improving liver histology comprises reducing the Metavir score by at least two grades or stages, e.g., reduction in liver fibrosis by at least two grades. In another embodiment, improving liver histology comprises reducing the Metavir score by three grades or stages, e.g., reduction in liver fibrosis by three grades.

One of the histopathological features associated with ALMS in the heart is reduced left ventricular function (LVF). Thus, in embodiments, improving heart histology comprises increasing left ventricular function (LVF), or slowing the progression of reduction of LVF. LVF may be determined using well-known methods and assays, including cardiac Magnetic Resonance Imaging (MRI). In an embodiment, increasing LVF comprises increasing left ventricular end-diastolic volume (LVEDV) and/or left ventricular end-systolic volume (LVESV). In an embodiment, increasing LVF comprises increasing left ventricular end-diastolic volume (LVEDV). In an embodiment, the method, formulation or use increases LVEDV by at least 6 ml relative to prior to the administration, as measured by cardiac MRI. In another embodiment, the method, formulation or use increases LVEDV by at least 6.5, 7, 7.5 or 8 ml relative to prior to the administration, as measured by cardiac MRI.

Another histopathological feature associated with ALMS in the heart is short-axis (plane) longitudinal relaxation time (SAX T1). Thus, in an embodiment, improving heart histology comprises reducing SAX T1, such as Basal SAX T1 and/or Mid SAX T1. SAX T1 may be determined using well-known methods and assays, including cardiac MRI. In an embodiment, the method, formulation or use is for reducing Basal SAX T1 by at least 40 ms relative to prior to the administration, as measured by cardiac MRI. In another embodiment, the method, formulation or use is for reducing Basal SAX T1 by at least 45, 50, 55 or 60 ms relative to prior to the administration, as measured by cardiac MRI. In an embodiment, the method, formulation or use is for reducing Mid SAX T1 by at least 25 ms relative to prior to the administration, as measured by cardiac MRI. In another embodiment, the method, formulation or use is for reducing Mid SAX T1 by at least 30, 35, 40 or 45 ms relative to prior to the administration, as measured by cardiac MRI.

One of the histopathological features associated with ALMS in the kidney is the presence of kidney damage or injury. Thus, in an embodiment, improving kidney histology comprises inhibiting or slowing the progression of kidney damage or injury. Kidney damage or injury may be determined using well-known methods and assays, such as by measuring the Albumin-to-creatinine ratio (ACR) or the levels of one or more biomarkers of kidney injury, such as monocyte chemoattractant protein 1 (MCP-1), kidney injury molecule-1 (KIM-1), clusterin, cystatin C and osteopontin. In an embodiment, the method, formulation or use is for inhibiting or slowing the increase of the ACR in the subject. In another embodiment, the method, formulation or use is for inhibiting or slowing the increase of the ACR in the subject. In another embodiment, the method, formulation or use is for reducing the levels of one or more biomarkers of kidney injury. In an embodiment, the levels of one or more of MCP-1, KIM-1, clusterin, cystatin C and osteopontin is reduced. In a further embodiment, the levels of MCP-1, KIM-1, clusterin, cystatin C and osteopontin is reduced. In an embodiment, the levels of the one or more biomarkers is reduced by at least 10% relative to prior to the treatment. In another embodiment, the levels of the one or more biomarkers is reduced by at least 15%, 20%, 25% or 30% relative to prior to the treatment.

One of the histopathological features associated with ALMS in adipose tissue is the hypertrophy and coalescence of adipocytes, forming giant vesicular vacuolation/steatosis. Thus, in an embodiment, improving adipose tissue histology comprises reducing average adipocyte area. In an embodiment, the method, formulation or use is for reducing average adipocyte area by at least 10% relative to prior to the administration. In another embodiment, the method, formulation or use is for reducing average adipocyte area by at least 15% or 20% relative to prior to the administration.

Adipose tissue artery (perivascular adipose tissue) in ALMS patients is characterized by signs of dystrophy, homogenized blurred cytoplasm in smooth muscle cells, and nuclei with irregular shape. Thus, in an embodiment, improving adipose tissue histology comprises restoring normal adipose tissue artery.

ALMS is also associated with several metabolic abnormalities. Insulin resistance, hyperinsulinemia, and impaired glucose tolerance often present in very early childhood. Type 2 diabetes (T2DM) develops in childhood, adolescence, or adulthood, with a mean age of onset at 16 years. Children with ALMS also often have high lipid levels at an early age. Thus, in an embodiment, improving a metabolic parameter comprises improving glycemic control. Glycemic control may be assessed using well-known methods and assays. In a further embodiment, improving glycemic control comprises reducing glycated hemoglobin (HbA1c). In an embodiment, the method, formulation or use is for reducing HbA1c by at least 0.5% or 1% (absolute).

In another embodiment, improving glycemic control comprises increasing the homeostasis Model Assessment for Steady State Beta-Cell Function Based on C-Peptide and Fasting Plasma Glucose (HOMA-B [C-Peptide/FPG]). In an embodiment, the method, formulation or use is for increasing HOMA-B [C-Peptide/FPG] by at least 20 relative to prior to the administration. In another embodiment, the method, formulation or use is for increasing HOMA-B [C-Peptide/FPG] by at least 25, 30 or 35 relative to prior to the administration.

In an embodiment, the ALMS patient is overweight, i.e. has a body mass index (BMI) 25.0 kg/m². In another embodiment, the ALMS patient is obese, i.e. has a BMI 30.0 kg/m².

In an embodiment, the ALMS patient is a male and has a waist circumference 94 cm. In another embodiment, the ALMS patient is a female and has a waist circumference 80 cm.

In an embodiment, the ALMS patient suffers from diabetes. In a further embodiment, the ALMS patient suffers from type 1 diabetes. In another embodiment, the ALMS patient suffers from type 2 diabetes.

In an embodiment, the ALMS patient has a Metavir Score (determined based on liver FibroScan results) of F2 or worse. In an embodiment, the ALMS patient has a Metavir Score (based on liver FibroScan results) of F3 or worse. In an embodiment, the ALMS patient has a Metavir Score (based on liver FibroScan results) of F4.

In an embodiment, the ALMS patient is a pediatric patient (17 years old or less). In another embodiment, the ALMS patient is an adult patient. In another embodiment, the ALMS patient is 18 to 52 years old, e.g. 20 to 35 years old.

In an embodiment, the 2-(3-pentylphenyl)acetate or pharmaceutically salt thereof is administered or for administration at a daily dose of about 500 mg to about 1500 mg, about 600 mg to about 1400 mg, about 700 mg to about 1300 mg, about 600 mg to about 1000 mg, about 700 mg to about 900 mg, about 750 mg to about 850 mg, about 775 mg to about 825 mg, about 900 mg to about 1500 mg, about 1000 mg to about 1400 mg, about 1100 mg to about 1300 mg, about 1150 mg to about 1250 mg, or about 11750 mg to about 1225 mg. In a further embodiment, the 2-(3-pentylphenyl)acetate or pharmaceutically salt thereof is administered or for administration at a daily dose of about 800 mg, about 900 mg, about 1000 mg, about 1100 mg, or about 1200 mg. The daily dose may be administered using a single dosage form or using a plurality of dosage forms (e.g., tablets, capsules). If the daily dose is administered using a plurality of dosage forms (e.g., 2, 3 or 4 dosage forms), the plurality of dosage forms may be taken at the same time or at different times during the day. In an embodiment, the plurality of dosage forms is taken at the same time.

In an embodiment, there is no treatment interruption of 20 days (e.g., consecutive days) or more during said treatment period. In an embodiment, there is no treatment interruption of 25 days (e.g., consecutive days) or more during said treatment period. In an embodiment, there is no treatment interruption of 30 days (e.g., consecutive days) or more during said treatment period. The term “treatment interruption” as used herein means a subject that does not take the above-noted daily dosage of 2-(3-pentylphenyl)acetate or pharmaceutically salt thereof for the specified number of days during the treatment period.

In an embodiment, the treatment period is at least 54 weeks. In another embodiment, the treatment period is at least 60 weeks.

The pharmaceutical oral formulation may be prepared by using standard methods known in the art by mixing the 2-(3-pentylphenyl)acetate or pharmaceutically salt thereof having the desired degree of purity with one or more pharmaceutically acceptable excipients (see Remington: The Science and Practice of Pharmacy, by Loyd V Allen, Jr., 2012, 22^{nd} edition, Pharmaceutical Press; Handbook of Pharmaceutical Excipients, by Rowe et al., 2012, 7^{th} edition, Pharmaceutical Press).

“Pharmaceutically acceptable excipient” as used herein refers to any excipient that does not interfere with effectiveness of the biological activity of the 2-(3-pentylphenyl)acetate or pharmaceutically salt thereof and that is not toxic to the subject, i.e., is a type of excipient and/or is for use in an amount which is not toxic to the subject. Excipients are well known in the art, and the present pharmaceutical oral formulation is not limited in these respects. In certain embodiments, the pharmaceutical oral formulation includes excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive.

Useful diluents, e.g., fillers, include, for example and without limitation, dicalcium phosphate, calcium diphosphate, calcium carbonate, calcium sulfate, lactose, cellulose, kaolin, sodium chloride, starches, powdered sugar, colloidal silicon dioxide, titanium oxide, alumina, talc, colloidal silica, microcrystalline cellulose, silicified micro crystalline cellulose and combinations thereof. Fillers that can add bulk to tablets with minimal drug dosage to produce tablets of adequate size and weight include croscarmellose sodium NF/EP (e.g., Ac-Di-Sol); anhydrous lactose NF/EP (e.g., Pharmatose™ DCL 21); and/or povidone USP/EP.

Binder materials include, for example and without limitation, starches (including corn starch and pregelatinized starch), gelatin, sugars (including sucrose, glucose, dextrose and lactose), polyethylene glycol, povidone, waxes, and natural and synthetic gums, e.g., acacia sodium alginate, polyvinylpyrrolidone, cellulosic polymers (e.g., hydroxypropyl cellulose, hydroxypropyl methylcellulose, methyl cellulose, hydroxyethyl cellulose, carboxymethylcellulose, colloidal silicon dioxide NF/EP (e.g., Cab-O-Sil™ M5P), Silicified Microcrystalline Cellulose (SMCC), e.g., Silicified microcrystalline cellulose NF/EP (e.g., Prosolv™ SMCC 90), and silicon dioxide, mixtures thereof, and the like), veegum, and combinations thereof.

Useful lubricants include, for example, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), DL-leucine, calcium stearate, sodium stearyl fumarate, mixtures thereof, and the like.

Bulking agents include, for example: microcrystalline cellulose, for example, AVICEL® (FMC Corp.) or EMCOCEL® (Mendell Inc.), which also has binder properties; dicalcium phosphate, for example, EMCOMPRESS® (Mendell Inc.); calcium sulfate, for example, COMPACTROL® (Mendell Inc.); and starches, for example, Starch 1500; and polyethylene glycols (CARBOWAX™).

Disintegrating or dissolution promoting agents include: starches, clays, celluloses, alginates, gums, crosslinked polymers, colloidal silicon dioxide, osmogens, mixtures thereof, and the like, such as crosslinked sodium carboxymethyl cellulose (AC-DI-SOL®), sodium croscarmelose, sodium starch glycolate (EXPLOTAB®, PRIMO JEL®) crosslinked polyvinylpolypyrrolidone (PLASONE-XL®), sodium chloride, sucrose, lactose and mannitol.

Antiadherents and glidants employable in the core and/or a coating of the solid oral dosage form may include talc, starches (e.g., cornstarch), celluloses, silicon dioxide, sodium lauryl sulfate, colloidal silica dioxide, and metallic stearates, among others.

Examples of silica flow conditioners include colloidal silicon dioxide, magnesium aluminum silicate and guar gum.

Suitable surfactants include pharmaceutically acceptable non-ionic, ionic and anionic surfactants. An example of a surfactant is sodium lauryl sulfate. If desired, the pharmaceutical composition to be administered may also contain minor amounts of nontoxic auxiliary substances such as wetting or emulsifying agents, pH-buffering agents and the like, for example, sodium acetate, sorbitan monolaurate, triethanolamine sodium acetate, triethanolamine oleate, etc. If desired, flavoring, coloring and/or sweetening agents may be added as well.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

Examples of thickening agents include talc USP/EP, a natural gum, such as guar gum or gum arabic, or a cellulose derivative such as microcrystalline cellulose NF/EP (e.g., Avicel™ PH 102), methylcellulose, ethylcellulose or hydroxyethylcellulose. A useful thickening agent is hydroxypropyl methylcellulose, an adjuvant which is available in various viscosity grades.

Examples of plasticizers include: acetylated monoglycerides; these can be used as food additives; Alkyl citrates, used in food packagings, medical products, cosmetics and children toys; Triethyl citrate (TEC); Acetyl triethyl citrate (ATEC), higher boiling point and lower volatility than TEC; Tributyl citrate (TBC); Acetyl tributyl citrate (ATBC), compatible with PVC and vinyl chloride copolymers; Trioctyl citrate (TOC), also used for gums and controlled release medicines; Acetyl trioctyl citrate (ATOC), also used for printing ink; Trihexyl citrate (THC), compatible with PVC, also used for controlled release medicines; Acetyl trihexyl citrate (ATHC), compatible with PVC; Butyryl trihexyl citrate (BTHC, trihexyl o-butyryl citrate), compatible with PVC; Trimethyl citrate (TMC), compatible with PVC; alkyl sulphonic acid phenyl ester, polyethylene glycol (PEG) or any combination thereof. Optionally, the plasticizer can comprise triethyl citrate NF/EP.

Examples of permeation enhancers include: sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol and polyethylene glycol), surfactants and terpenes.

Formulations suitable for oral administration include capsules, sachets or tablets, each containing a predetermined amount of the active ingredient, as liquids, solids, granules or gelatin. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

In an embodiment, the pharmaceutical oral formulation is a capsule or a tablet. In an embodiment, the pharmaceutical oral formulation is a capsule. In another embodiment, the pharmaceutical oral formulation is a tablet.

### EXAMPLES

The present disclosure is illustrated in further details by the following non-limiting examples.

### Example 1: Details of Clinical Study

Methods

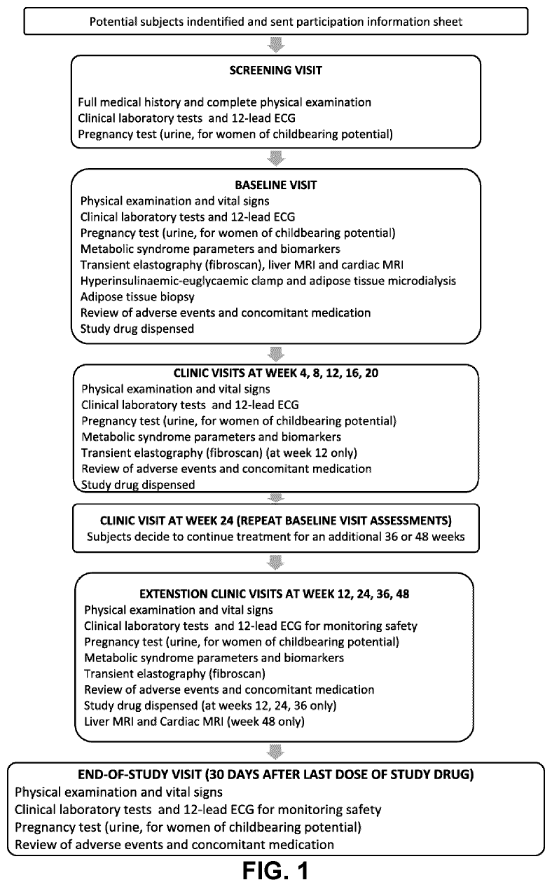

The study was a Phase 2, single-centre, single-arm, open-label study of sodium 2-(3-pentylphenyl)acetate administered at a total daily oral dose of 800 mg for up to 72 weeks in subjects with ALMS was undertaken. The initial duration of the study was 24 weeks, and subjects were offered to enrol into an extension phase of 36 or 48 weeks. The schedule of study procedures for the enrolment, intervention, and assessments for participants is outlined in FIG. 1.

Sodium 2-(3-pentylphenyl)acetate was formulated in soft gelatine capsules containing 200 mg of Sodium 2-(3-pentylphenyl)acetate (amorphous powder) per capsule, polyethylene glycol 400, National Formulary (NF), hydrochloric acid (for pH adjustment), and water.

Study Population and Eligibility Criteria

A total of 15 subjects participated in the study. Three subjects did not meet eligibility criteria and were not enrolled into the study (i.e., screen failures). Twelve (12) subjects met the eligibility criteria and received at least 1 dose of 800 mg sodium 2-(3-pentylphenyl)acetate, with all 12 subjects completing the initial 24-week treatment period as planned. A 36-week EP was added to the study protocol (Table 1). Ten (10) of the 12 subjects who completed the initial 24-week study drug treatment period continued in the EP, with 6 subjects having a break in treatment until the amendment was implemented before continuing treatment in the EP and 4 subjects continuing in the EP without any break in treatment. Two subjects (826-001-002 and 826-001-014) completed the study after the initial 24-week study drug treatment period.

TABLE 1
Listing of Subjects Who Continued Study Drug
Treatment in the Extension Period

| | Break in | Duration of |
|---|---|---|
| | Treatment | Treatment |
| Subject ID | (Yes or No) | Break |
| 826-001-001 | Yes | 204 days |
| 826-001-003 | Yes | 32 days |
| 826-001-004 | Yes | 30 days |
| 826-001-005 | Yes | 39 days |
| 826-001-006 | No | NA |
| 826-001-007 | Yes | 32 days |
| 826-001-009 | No | NA |
| 826-001-012 | No | NA |
| 826-001-013 | Yes | 30 days |
| 826-001-015 | No | NA |

ID = identification number;
NA = not applicable.

Of the 10 subjects continuing study drug treatment in the EP, all but 1 subject completed the study as planned.

The mean age (range) of the subject population was 26.1 years (17-52 years), with most subjects being male and White (66.7%), and none of the subjects were Hispanic or Latino (Table 2). Half of the subjects were obese (body mass index [BMI]≥30.0 kg/m²), and all but 1 subject was at least overweight (BMI 25.0 kg/m²). In addition, most subjects had a waist circumference at increased risk of health problems (males 94 cm and females 80 cm).

TABLE 2
Baseline Demographic Characteristics

| | | | | | | Waist | | |
|---|---|---|---|---|---|---|---|---|
| | | | | | | Circum- | | |
| | | | | | Weight | ference | Height | BMI |
| Subject ID | Age | Gender | Ethnicity | Race | (kg) | (cm) | (cm) | (kg/m²) |
| 826-001-001 | 20 | Male | NHL | White | 69.6 | 94.0 | 165.0 | 25.56 |
| 826-001-002 | 31 | Male | NHL | White | 82.1 | 97.0 | 159.0 | 32.47 |
| 826-001-003 | 24 | Female | NHL | White | 83.4 | 99.0 | 164.0 | 31.01 |
| 826-001-004 | 25 | Female | NHL | White | 58.4 | 87.5 | 152.8 | 25.01 |
| 826-001-005 | 22 | Male | NHL | White | 72.6 | 87.5 | 166.0 | 26.35 |
| 826-001-006 | 20 | Female | NHL | Asian | 104.0 | 125.0 | 151.0 | 45.61 |
| 826-001-007 | 20 | Male | NHL | Asian | 86.1 | 99.0 | 166.0 | 31.25 |
| 826-001-009 | 24 | Male | NHL | White | 87.9 | 105.5 | 170.0 | 30.42 |
| 826-001-012 | 52 | Male | NHL | White | 85.8 | 105.0 | 150.0 | 38.13 |
| 826-001-013 | 17 | Male | NHL | Asian | 69.9 | 85.0 | 165.0 | 25.67 |
| 826-001-014 | 23 | Male | NHL | Asian | 62.9 | 86.0 | 150.0 | 27.96 |
| 826-001-015 | 35 | Female | NHL | White | 53.1 | 82.0 | 146.5 | 24.74 |

BMI = body mass index;
ID = identification number;
NHL = not Hispanic/Latino.

The median duration (range) of ALMS was 13.2 years (3-22 years), and most subjects had type 2 diabetes (T2D; 83.3%), significant liver fibrosis (Metavir score 2; 72.7%), and kidney disease (75.0%; Table 3). Of note, Subject 826-001-003 had both type 1 diabetes (T1D) and T2D; Subjects 826-001-007 and 826-001-015 had possible cirrhosis (based on FibroScan® results); and Subject 826-001-012 had possible heart failure.

TABLE 3
Baseline Characteristics

| | | | Metavir | | |
|---|---|---|---|---|---|
| | | | Score Based | NT- | |
| | ALMS | | on Liver | pro- | |
| | Duration^{a} | Diabetes | FibroScan ® | BNP^{c} | ACR^{d} |
| Subject ID | (months) | Status | Results^{b} | (ng/L) | (mg/mmol) |
| 826-001-001 | 169.7 | Type 2 | F3 | 34 | 6.3 |
| 826-001-002 | 159.3 | Type 2 | F2 | <10 | 17.6 |
| 826-001-003 | 183.3 | Type 1/2 | F0/1 | 59 | 13.7 |
| 826-001-004 | 106.7 | Type 2 | F2 | 68 | 17.7 |
| 826-001-005 | 171.8 | None | F0/1 | 8 | 0.7 |
| 826-001-006 | 38.5 | None | F2 | 59 | 4.4 |
| 826-001-007 | 52.2 | Type 2 | F4 | 34 | 44.5 |
| 826-001-009 | 156.8 | Type 2 | F0/1 | <10 | 1.7 |
| 826-001-012 | 59.2 | Type 2 | F3 | 279 | 6.9 |
| 826-001-013 | 65.7 | Type 2 | F3 | <10 | 41.5 |
| 826-001-014 | 266.8 | Type 2 | — | 51 | 18.6 |
| 826-001-015 | 183.3 | Type 2 | F4 | 25 | 1.1 |

— = no data available;
ACR = albumin/creatinine ratio;
ALMS = Alström syndrome;
ICF = informed consent form;
ID = identification number;
NT-pro-BNP = N-terminal pro-brain natriuretic peptide.
^{a}ALMS duration was measured from the diagnosis date to the ICF date.
^{b}The Metavir scoring system is a tool used to evaluate the severity of liver fibrosis using transient elastography (FibroScan ®). Metavir score F0-F1: absence or mild fibrosis (2.5-7.0 kPa on FibroScan ®); Metavir score F2: significant fibrosis (≥7.1 to <9.5 kPa on FibroScan); Metavir Score F3: severe fibrosis (≥9.5 to <12.5 kPa on FibroScan ®; and Metavir score F4: cirrhosis (≥12.5 kPa on FibroScan ®).
^{c}Normal range: <125 ng/L in subjects 0-74 years of age.
^{d}ACR < 3.39 mg/mmol = normal; ACR 3.39 to 33.9 mg/mmol = early kidney disease (microalbuminuria); and ACR > 33.9 mg/mmol = more advanced kidney disease (macroalbuminuria).

### Example 2: Effect of 800 mg Sodium 2-(3-Pentylphenyl)Acetate on Liver

There is phenotypic variation in the slowly progressive hepatic dysfunction in Alström syndrome, which begins with clinically silent elevation of transaminases, and steatosis. The initial presentation is usually steatosis and hepatosplenomegaly followed by fibrotic and inflammatory processes with lymphocytic infiltration in the portal and parenchymal areas. In the final course of hepatic disease, there is significant fibrosis, cirrhosis, and portal hypertension (Marshall et al., Curr Genomics. 2011 May; 12(3): 225-235).

A) Liver Stiffness

The effect of sodium 2-(3-pentylphenyl)acetate on liver histology in ALMS patients was first assessed by measuring liver stiffness. Liver stiffness is a physical parameter that reflects the health of the liver and is commonly used to diagnose liver diseases. Increased liver stiffness may be associated with hepatic dysfunction.

Statistically significant decreases in liver stiffness from baseline, as measured by transient elastography via FibroScan®, were seen after 60 weeks of treatment with sodium 2-(3-pentylphenyl)acetate (2.59 kPa; p=0.0207; Table 4).

TABLE 4
Effect of 800 mg sodium 2-(3-pentylphenyl)acetate on the Change from Baseline in
Liver Stiffness Using Transient Elastography (FibroScan ®): Change from
Baseline Transient Elastography (kPa)

| | Change from Baseline Transient Elastography (kPa) | | | | |
|---|---|---|---|---|---|
| | | | EP Week 12 | EP Week 24 | EP Week 36 |
| Statistics | Week 12 | Week 24 | (Week 36) | (Week 48) | (Week 60) |
| N | 11 | 11 | 10 | 10 | 9 |
| Mean (SD) | −1.19 (2.160) | −1.01 (2.308) | −1.41 (2.585) | −1.49 (2.335) | −2.59 (2.703) |
| Median | −1.50 | −0.70 | −1.75 | −1.45 | −2.30 |
| PT P-value | 0.0947 | 0.1777 | 0.1187 | 0.0744 | 0.0207 |
| PT 95% CI | −2.64, 0.26 | −2.56, 0.54 | −3.26, 0.44 | −3.16, 0.18 | −4.67, −0.51 |
| SW P-value | 0.5017 | 0.4882 | 0.4217 | 0.9457 | 0.7706 |
| WS P-value | 0.1055 | 0.1416 | 0.0840 | 0.0977 | 0.0195 |

CI = confidence interval;
EP = Extension Period;
PT = paired t-test;
SD = standard deviation;
SW = Shapiro-Wilk test;
WS = Wilcoxon signed-rank test.

Liver stiffness was variable at baseline, ranging from 5.1 to 21.1 kPa (Table 5), with the lowest baseline liver stiffness occurring in a non-diabetic subject with no medical history of liver disease (826-001-005) and the highest baseline liver stiffness occurring in a T2D subject with an ongoing medical history of abnormal hepatic function (826-001-007). Liver stiffness decreased from baseline in the majority of subjects (8/11, 72.3%) after study drug treatment with 800 mg sodium 2-(3-pentylphenyl)acetate. Decreases in liver stiffness from baseline ranged from 0.6 to 8.0 kPa. Of note, a total of 4 subjects (826-001-001, 826-001-004, 826-001-012, and 826-001-015) had at least a 1-grade reduction in liver fibrosis, as assessed by the Metavir scoring system, after study drug treatment. This included 1 subject (826-001-015) whose Metavir score decreased from F4 (baseline) to F0/1 (Week 60). The largest increase in liver stiffness from baseline occurred in Subject 826-001-013 (1.8 kPa), who had a 30-day break in study drug treatment between Week 24 and the start of the EP.

TABLE 5
Effect of 800 mg sodium 2-(3-pentylphenyl)acetate on Liver
Stiffness Using Transient Elastography (FibroScan ®): Transient
Elastography Score (kPa) and Metavir Score (F) - individual results

| | Transient Elastography Score (kPa) | | | | | |
|---|---|---|---|---|---|---|
| | and Metavir Score (F) ^{a} | | | | | |
| | | | | EP | EP | EP |
| | | | | Week | Week | Week |
| | | | | 12 | 24 | 36 |
| | | Week | Week | (Week | (Week | (Week |
| Subject ID | Baseline | 12 | 24 | 36) | 48) | 60) |
| 826-001-001^{b, c} | 9.9 | 7.1 | 7.2 | 6.6 | 7.1 | 7.8 |
| | (F3) | (F2) | (F2) | (F0/1) | (F2) | (F2) |
| 826-001-002^{c} | 7.4 | 6.1 | 7.6 | — | — | — |
| | (F2) | (F0/1) | (F2) | | | |
| 826-001-003^{d, e} | 6.1 | 4.5 | 5.9 | 4.2 | 4.1 | 3.8 |
| | (F0/1) | (F0/1) | (F0/1) | (F0/1) | (F0/1) | (F0/1) |
| 826-001-004^{f} | 8.1 | 5.3 | 5.2 | 6.5 | 4.7 | 5.0 |
| | (F2) | (F0/1) | (F0/1) | (F0/1) | (F0/1) | (F0/1) |
| 826-001-005^{c, g, h} | 5.1 | 3.1 | 4.6 | 4.6 | 5.1 | 3.9 |
| | (F0/1) | (F0/1) | (F0/1) | (F0/1) | (F0/1) | (F0/1) |
| 826-001-006^{c, h} | 8.8 | 10.5 | 8.1 | 8.9 | 7.5 | — |
| | (F2) | (F3) | (F2) | (F2) | (F2) | |
| 826-001-007^{c, d} | 21.1 | 19.6 | 21.8 | 18.6 | 21.6 | 17.1 |
| | (F4) | (F4) | (F4) | (F4) | (F4) | (F4) |
| 826-001-009^{c} | 6.6 | 7.8 | 5.6 | 5.8 | 5.0 | 6.0 |
| | (F0/1) | (F2) | (F0/1) | (F0/1) | (F0/1) | (F0/1) |
| 826-001-012 | 12.4 | 12.2 | 8.6 | 10.2 | 12.1 | 8.6 |
| | (F3) | (F3) | (F2) | (F3) | (F3) | (F2) |
| 826-001-013^{f} | 10.4 | 12.0 | 14.3 | 14.7 | 12.6 | 12.2 |
| | (F3) | (F3) | (F4) | (F4) | (F4) | (F3) |
| 826-001-014^{c, e} | — | 50.5 | 48.9 | — | — | — |
| | | (F4) | (F4) | | | |
| 826-001-015^{c} | 13.1 | 7.7 | 9.0 | 7.4 | 6.9 | 5.1 |
| | (F4) | (F2) | (F2) | (F2) | (F0/1) | (F0/1) |

— = no data available; EP = Extension Period; ID = identification number.
^{a} The Metavir scoring system is a tool used to evaluate the severity of liver fibrosis usingtransient elastography (FibroScan). Metavir score F0-F1: absence or mild fibrosis (2.5-7.0 kPa on FibroScan ®); Metavir score F2: significant fibrosis ≥ 7.1 to < 9.5 kPa on FibroScan ®); Metavir score F3: severe fibrosis ≥ 9.5 to < 12.5 kPa on FibroScan ®; and Metavir score F4: cirrhosis( ≥ 12.5 kPa on FibroScan ®).
^{b}204-day break in study drug treatment between Week 24 and EP Day 0 (first day of EP study drug treatment).
^{c}Subject had ongoing medical history of liver disorder (e.g., hepatic steatosis, liver disorder, and/or hepatic function abnormal).
^{d}32-day break in study drug treatment between Week 24 and EP Day 0 (first day of EP study drug treatment).
^{e}Insulin-dependent T2D subject.
^{f}30-day break in study drug treatment between Week 24 and EP Day 0 (first day of EP study drug treatment).
^{g}Non-diabetic.
^{h}39-day break in study drug treatment between Week 24 and EP Day 0 (first day of EP study drug treatment).

These results provide evidence that treatment with sodium 2-(3-pentylphenyl)acetate for a period of more than 48 weeks, and preferably for at least 60 weeks, leads to significant improvement of liver histology in ALMS patients, and that treatment interruption may lead to deterioration of liver histology.

B) NAFLD Fibrosis Score

The effect of sodium 2-(3-pentylphenyl)acetate on liver histology in ALMS patients was also assessed by measuring the Non-Alcoholic Fatty Liver Disease (NAFLD) Fibrosis Score. Increased NAFLD fibrosis score may be associated with hepatic dysfunction.

The results are depicted in Table 6.

TABLE 6
Effect of 800 mg sodium 2-(3-pentylphenyl)acetate on Non-Alcoholic
Fatty Liver Disease Fibrosis Score

| | Change from Baseline NAFLD Score with an FPG > 5.6 mmol/L ^{a} | | | | |
|---|---|---|---|---|---|
| | | | EP Week 12 | EP Week 24 | EP Week 36 |
| Statistics | Week 12 | Week 24 | (Week 36) | (Week 48) | (Week 60) |
| N | 11 | 9 | 10 | 9 | 9 |
| Mean (SD) | −0.35 (0.582) | 0.16 (0.446) | −0.39 (0.683) | −0.34 (0.758) | −0.16 (0.684) |
| Median | −0.34 | 0.21 | −0.69 | −0.28 | −0.14 |
| PT P-value | 0.0706 | 0.3067 | 0.1039 | 0.2155 | 0.5097 |
| PT 95% CI | −0.75, 0.04 | −0.18, 0.51 | −0.88, 0.10 | −0.92, 0.24 | −0.68, 0.37 |
| SW P-value | 0.1911 | 0.8053 | 0.2399 | 0.0295 | 0.8666 |
| WS P-value | 0.0674 | 0.4961 | 0.1309 | 0.2031 | 0.7344 |

CI = confidence interval;
EP = Extension Period;
FPG = fasting plasma glucose;
NAFLD = non-alcoholic fatty liver disease;
NASH = non-alcoholic steatohepatitis;
PT = paired t-test;
SD = standard deviation;
SW = Shapiro-Wilk test;
WS = Wilcoxon signed-rank test.
^{a} NAFLD Fibrosis Score: <−1.455 (low likelihood of NASH); −1.455 to 0.676 (indeterminate likelihood of NASH); >0.676 (high likelihood of NASH).

Baseline NAFLD fibrosis scores ranged from −4.6 to 0.9 in subjects with either a baseline FPG>5.6 mmol/L or >6.0 mmol/L (Table 7), with most subjects (66.7%) having an NAFLD fibrosis score associated with a low likelihood of non-alcoholic steatohepatitis. NAFLD fibrosis scores decreased or did not change from baseline in the majority of subjects (7/12, 58.3%) after study drug treatment with 800 mg sodium 2-(3-pentylphenyl)acetate. Of note, an increase in NAFLD fibrosis score from baseline occurred in Subjects 826-001-001 (0.7), who had a 204-day break in study drug treatment between Week 24 and the start of the EP.

TABLE 7
Effect of 800 mg sodium 2-(3-pentylphenyl)acetate on Non-Alcoholic
Fatty Liver Disease Fibrosis Score - individual results

| | NAFLD Fibrosis Score in Subjects with an | | | | | |
|---|---|---|---|---|---|---|
| | FPG > 5.6 mmol/L^{a} | | | | | |
| | | | | EP | EP | EP |
| | | | | Week | Week | Week |
| | | | | 12 | 24 | 36 |
| | | Week | Week | (Week | (Week | (Week |
| Subject ID | Baseline | 12 | 24 | 36) | 48) | 60) |
| 826-001-001^{b, c} | −2.2 | −2.5 | −1.5 | −1.9 | −1.9 | −1.5 |
| 826-001-002^{c} | −1.8 | −1.8 | −1.9 | — | — | — |
| 826-001-003^{d, e} | −2.3 | −3.9 | −2.7 | −3.1 | −4.4 | −3.7 |
| 826-001-004^{f} | −2.9 | −3.1 | −2.7 | −3.8 | −3.0 | −2.9 |
| 826-001-005^{c, g, h} | −4.1 | −3.9 | −3.8 | −3.8 | −3.8 | −4.2 |
| 826-001-006^{c, h} | −0.5 | −1.4 | −0.2 | −1.6 | −0.8 | — |
| 826-001-007^{c, d} | −0.5 | −0.8 | — | −1.2 | −1.0 | −1.1 |
| 826-001-009^{c} | −2.5 | −3.3 | −2.9 | −3.8 | −3.1 | −2.9 |
| 826-001-012 | 0.9 | 0.6 | — | 0.2 | 0.5 | 0.2 |
| 826-001-013^{f} | −3.7 | — | — | −3.6 | — | −3.4 |
| 826-001-014^{c, e} | −1.0 | −0.8 | −1.0 | — | — | — |
| 826-001-015^{c} | −4.6 | −4.3 | −3.7 | −3.8 | −4.2 | −3.8 |

— = no data available; EP = Extension Period; FPG = fasting plasma glucose; ID = identification number; NAFLD = non-alcoholic fatty liver disease; NASH = non-alcoholic steatohepatitis.
^{a}NAFLD Fibrosis Score: < −1.455 (low likelihood of NASH); −1.455 to 0.676 (indeterminate likelihood of NASH); > 0.676 (high likelihood of NASH).
^{b}204-day break in study drug treatment between Week 24 and EP Day 0 (first day of EP study drug treatment).
^{c}Subject had ongoing medical history of liver disorder (e.g., hepatic steatosis, liver disorder, and/or hepatic function abnormal).
^{d}32-day break in study drug treatment between Week 24 and EP Day 0 (first day of EP study drug treatment).
^{e}Insulin-dependent T2D subject.
^{f}30-day break in study drug treatment between Week 24 and EP Day 0 (first day of EP study drug treatment).
^{g}Non-diabetic.
^{h}39-day break in study drug treatment between Week 24 and EP Day 0 (first day of EP study drug treatment).

C) Enhanced Liver Fibrosis Test

The effect of sodium 2-(3-pentylphenyl)acetate on liver histology in ALMS patients was also assessed using the Enhanced Liver Fibrosis (ELF™) blood test. The ELF™ Blood Test combines three serum biomarkers (Hyaluronic acid (HA), Procollagen III amino terminal peptide (PIIINP), and Tissue inhibitor of metalloproteinase 1 (TIMP-1)) which, when correlated, are able to identify a quantifiable level of liver fibrosis.

The results (changes from baseline) are depicted in Table 8.

TABLE 8
Effect of 800 mg PBI-4050 on the Change from Baseline in the Enhanced Liver Fibrosis Test

| | Change from Baseline ELF Test | | | | |
|---|---|---|---|---|---|
| | | | EP Week 12 | EP Week 24 | EP Week 36 |
| Statistics | Week 12 | Week 24 | (Week 36) | (Week 48) | (Week 60) |
| N | 12 | 12 | 10 | 9 | 8 |
| Mean (SD) | 0.02 (1.023) | −0.26 (0.933) | −0.24 (1.056) | −0.22 (1.086) | −0.26 (1.193) |
| Median | 0.24 | 0.06 | −0.02 | −0.13 | −0.03 |
| PT P-value | 0.9472 | 0.3564 | 0.4923 | 0.5565 | 0.5551 |
| PT 95% CI | −0.63, 0.67 | −0.85, 0.33 | −0.99, 0.52 | −1.06, 0.61 | −1.26, 0.74 |
| SW P-value | 0.3329 | 0.5798 | 0.2689 | 0.6739 | 0.6982 |
| WS P-value | 0.7334 | 0.5186 | 0.6250 | 0.5703 | 0.7422 |

CI = confidence interval;
ELF = Enhanced Liver Fibrosis;
EP = Extension Period;
PT = paired t-test;
SD = standard deviation;
SW = Shapiro-Wilk test;
WS = Wilcoxon signed-rank test.

Baseline ELF test scores ranged from 7.11 and 11.65, with most subjects (66.7%) having moderate fibrosis (Table 9). ELF test scores decreased from baseline in half the subjects (6/12, 50.0%) after study drug treatment. Decreases in ELF test scores from baseline ranged from 0.1 to 1.75. Of note, the largest increases in ELF test scores from baseline occurred in

Subjects 826-001-001 (1.36) and 826-001-004 (1.17), who had a break in study drug treatment (204 and 30 days for Subjects 826-001-001 and 826-001-004, respectively) between Week 24 and the start of the EP.

TABLE 9
By-Subject Listing of the Enhanced Liver Fibrosis Test

| | ELF Test ^{a} | | | | | |
|---|---|---|---|---|---|---|
| | | | | EP | EP | EP |
| | | | | Week | Week | Week |
| | | | | 12 | 24 | 36 |
| | | Week | Week | (Week | (Week | (Week |
| Subject ID | Baseline | 12 | 24 | 36) | 48) | 60) |
| 826-001-001^{b,c} | 8.92 | 8.50 | 9.31 | 9.13 | 10.30 | 10.28 |
| 826-001-002^{c} | 7.93 | 8.63 | 9.02 | — | — | — |
| 826-001-003^{d,e} | 8.04 | 9.75 | 8.60 | 8.68 | 8.00 | 8.42 |
| 826-001-004^{f} | 7.11 | 7.98 | 7.14 | 7.68 | 6.98 | NA |
| 826-001-005^{c,g} | 9.30 | 7.93 | 7.26 | 8.19 | 7.45 | 7.16 |
| 826-001-006^{c,h} | 8.70 | 7.33 | 7.31 | 6.80 | 6.95 | — |
| 826-001-007^{c,d} | 11.65 | 10.05 | 10.39 | 9.92 | NA | 9.90 |
| 826-001-009^{c} | 9.06 | 9.71 | 8.41 | 8.48 | 8.36 | 8.50 |
| 826-001-012 | 10.59 | 10.96 | 10.69 | 11.58 | 11.14 | 10.79 |
| 826-001-013^{f} | 7.97 | 8.51 | 8.47 | 8.75 | 8.76 | 8.64 |
| 826-001-014^{c,e} | 11.15 | 11.25 | 11.29 | — | — | — |
| 826-001-015^{c} | 8.99 | 9.05 | 8.41 | 8.73 | 8.74 | 8.74 |

— = no data available; ELF = Enhanced Liver Fibrosis; EP = Extension Period; ID = identification number; NA = not available.
^{a} ELF test score: None to mild fibrosis < 7.7; moderate fibrosis 7.7 to < 9.8; and severe fibrosis 9.8.
^{b}204-day break in study drug treatment between Week 24 and EP Day 0 (first day of EP study drug treatment).
^{c}Subject had ongoing medical history of liver disorder (e.g., hepatic steatosis, liver disorder, and/or hepatic function abnormal).
^{d}32-day break in study drug treatment between Week 24 and EP Day 0 (first day of EP study drug treatment).
^{e}Insulin-dependent T2D subject.
^{f}30-day break in study drug treatment between Week 24 and EP Day 0 (first day of EP study drug treatment).
^{g}Non-diabetic.
^{h}39-day break in study drug treatment between Week 24 and EP Day 0 (first day of EP study drug treatment).

The severity of liver fibrosis based on the ELF test was stable or improved from baseline in the majority of subjects (10/12, 83.3%) after treatment (Table 10). An increase in liver fibrosis from moderate to severe was detected in Subjects 826-001-001, who had a 204-day break in study drug treatment.

TABLE 10
Effect of 800 mg sodium 2-(3-pentylphenyl)acetate on the
Severity of Liver Fibrosis - individual results

| | Severity of Liver Fibrosis Based on ELF Test ^{a} | | | | | |
|---|---|---|---|---|---|---|
| | | | | EP | EP | EP |
| | | | | Week | Week | Week |
| | | | | 12 | 24 | 36 |
| | | Week | Week | (Week | (Week | (Week |
| Subject ID | Baseline | 12 | 24 | 36) | 48) | 60) |
| 826-001-001^{b,c} | Mod | Mod | Mod | Mod | Sev | Sev |
| 826-001-002^{c} | Mod | Mod | Mod | — | — | — |
| 826-001-003^{d,e} | Mod | Mod | Mod | Mod | Mod | Mod |
| 826-001-004^{f} | NTM | Mod | NTM | NTM | NTM | NA |
| 826-001-005^{c,g,h} | Mod | Mod | NTM | Mod | NTM | NTM |
| 826-001-006^{c,h} | Mod | NTM | NTM | NTM | NTM | — |
| 826-001-007^{c,d} | Sev | Sev | Sev | Sev | NA | Sev |
| 826-001-009^{c} | Mod | Mod | Mod | Mod | Mod | Mod |
| 826-001-012 | Sev | Sev | Sev | Sev | Sev | Sev |
| 826-001-013^{f} | Mod | Mod | Mod | Mod | Mod | Mod |
| 826-001-014^{c,e} | Sev | Sev | Sev | — | — | — |
| 826-001-015^{c} | Mod | Mod | Mod | Mod | Mod | Mod |

— = no data available; ELF = Enhanced Liver Fibrosis; EP = Extension Period; ID = identification number; NTM = normal to mild; Mod = moderate; Sev = severe.
^{a} ELF test score: None to mild fibrosis < 7.7; moderate fibrosis ≥ 7.7 to < 9.8; and severe fibrosis ≥ 9.8.
^{b}204-day break in study drug treatment between Week 24 and EP Day 0 (first day of EP study drug treatment).
^{c}Subject had ongoing medical history of liver disorder (e.g., hepatic steatosis, liver disorder, and/or hepatic function abnormal).
^{d}32-day break in study drug treatment between Week 24 and EP Day 0 (first day of EP study drug treatment).
^{e}Insulin-dependent T2D subject.
^{f}30-day break in study drug treatment between Week 24 and EP Day 0 (first day of EP study drug treatment).
^{g}Non-diabetic.
^{h}39-day break in study drug treatment between Week 24 and EP Day 0 (first day of EP study drug treatment).

These results provide evidence that treatment with sodium 2-(3-pentylphenyl)acetate for a period of more than 48 weeks, and preferably for at least 60 weeks, leads to significant improvement of liver histology (more specifically liver stiffness) in ALMS patients, and that treatment interruption is associated with deterioration of liver histology, as assessed using various parameters.

### Example 3: Effect of 800 mg Sodium 2-(3-Pentylphenyl)Acetate on Heart Histology

Cardiomyopathy is a well-recognized feature in infants as well as in older children and adults with ALMS. Histopathology and cardiac Magnetic Resonance Imaging (MRI) analyses have revealed interstitial fibrosis affecting the myocardium in ALMS patients (Brofferio et al., Mol Genet Metab. 2017 August; 121(4): 336-343).

A) Cardiac Magnetic Resonance Imaging of Left Ventricular Function

The effect of sodium 2-(3-pentylphenyl)acetate on heart histology in ALMS patients was assessed by measuring left ventricular function (LVF). LVF measurements permit to quantify how well the left ventricle is able to pump blood through the body with each heartbeat, and is also a prognostic factor in acute myocardial infarction.

The results depicted in Table 11A demonstrate a statistically significant mean increase of 7.75 mL (p=0.0294) in left ventricular end-diastolic volume (LVEDV), and a strong trend toward an increase in left ventricular end-systolic volume (LVESV), from baseline after 60 weeks of treatment.

TABLE 11A
Effect of 800 mg sodium 2-(3-pentylphenyl)acetate on Magnetic
Resonance Imaging Assessment of LVESV and LVEDV

| | Change from Baseline | | | |
|---|---|---|---|---|
| | LVESV (mL) | | LVEDV (mL) | |
| | | EP Week 36 | | EP Week 36 |
| Statistics | Week 24 | (Week 60) | Week 24 | (Week 60) |
| N | 10 | 4 | 10 | 4 |
| Mean (SD) | 0.70 (9.650) | 3.75 (2.500) | 4.40 (16.574) | 7.75 (3.948) |
| Median | 3.00 | 3.50 | 5.00 | 8.50 |
| PT P-value | 0.8237 | 0.0577 | 0.4229 | 0.0294 |
| PT 95% CI | −6.20, 7.60 | −0.23, 7.73 | −7.46, 16.26 | 1.47, 14.03 |
| SW P-value | 0.6121 | 0.9109 | 0.1927 | 0.2498 |
| WS P-value | 0.8340 | 0.1250 | 0.5156 | 0.1250 |

CI = confidence interval;
LVEDV = left ventricular end-diastolic volume;
LVESV = left ventricular end-systolic volume;
PT = paired t-test;
SD = standard deviation;
SW = Shapiro-Wilk test;
WS = Wilcoxon signed-rank test.

Cardiac MRI of left ventricular function was variable at baseline (Table 11B). LVEDV increased from baseline in the majority of subjects (10/12, 83.3%) after treatment, with increases ranging from 2 to 42 mL. LVESV increased or did not change from baseline in the majority of subjects (9/12, 75.0%) after treatment, with increases ranging from 1 to 15 mL. Interestingly, subjects 826-001-002 and 826-001-004 had LVEDV and LVESV below normal at baseline, but within the normal range after the treatment period. Normalization of LVESV after treatment also occurred in subject 826-001-015, which suggests that sodium 2-(3-pentylphenyl)acetate may be useful for normalizing left ventricular function in ALMS patients with impaired (i.e. lower than normal) left ventricular function (e.g., LVEDV and/or LVESV).

B) Cardiac Magnetic Resonance Imaging of Myocardial Fibrosis

The effect of sodium 2-(3-pentylphenyl)acetate on heart histology in ALMS patients was also assessed by measuring short-axis (plane) longitudinal relaxation time (SAX T1). A decrease in relaxation time reflects an improvement in heart histology, such as a decrease of myocardial fibrosis, which reduces the risk of cardiac failure.

The results depicted in Table 12 demonstrate statistically significant mean decreases in Basal SAX T1 (84.75 ms; p=0.0274) and Mid SAX T1 (54.63 ms; p=0.0349) from baseline after 60 weeks of treatment with sodium 2-(3-pentylphenyl)acetate.

TABLE 12
Effect of 800 mg sodium 2-(3-pentylphenyl)acetate on
Magnetic Resonance Imaging Assessment of SAX T1

| | Change from Baseline | | | |
|---|---|---|---|---|
| | Basal SAX T1 (ms) | | Mid SAX T1 (ms) | |
| | | EP | | EP |
| | | Week 36 | | Week 36 |
| Statistics | Week 24 | (Week 60) | Week 24 | (Week 60) |
| N | 9 | 4 | 9 | 4 |
| Mean | −10.67 | −84.75 | −4.11 | −54.63 |
| (SD) | (44.172) | (42.003) | (24.427) | (29.722) |
| Median | −1.50 | −80.50 | 0.00 | −51.00 |
| PT P- | 0.4894 | 0.0274 | 0.6272 | 0.0349 |
| value | | | | |
| PT 95% | −44.62, | −151.59, | −22.89, | −101.92, |
| CI | 23.29 | −17.91 | 14.67 | −7.33 |
| SW P- | 0.3835 | 0.9331 | 0.4701 | 0.8985 |
| value | | | | |
| WS P- | 0.4961 | 0.1250 | 0.7422 | 0.1250 |
| value | | | | |

### Example 4: Effect of 800 mg Sodium 2-(3-Pentylphenyl)Acetate on Kidney Histology

Renal disease is a hallmark of ALMS, starting early and progressing with age, leading to a high prevalence of advanced chronic kidney disease (CKD) at young age in ALMS patients (Baig et al., Nephrol Dial Transplant. 2018 Oct. 10. doi: 10.1093/ndt/gfy293. [Epub ahead of print]).

A) Albumin-to-Creatinine Ratio (ACR)

Albuminuria, the increase in excretion of urinary albumin, is marker of kidney damage and is assessed by measuring the ACR. The effect of sodium 2-(3-pentylphenyl)acetate on kidney histology/damage in ALMS patients was assessed by measuring the ACR. The results reported in Table 13.

TABLE 13
Effect of 800 mg sodium 2-(3-pentylphenyl)acetate on ACR (change from baseline)

| | Change from Baseline ACR (mg/mmol) | | | | |
|---|---|---|---|---|---|
| | | | EP Week 12 | EP Week 24 | EP Week 36 |
| Statistics | Week 12 | Week 24 | (Week 36) | (Week 48) | (Week 60) |
| N | 12 | 11 | 10 | 10 | 9 |
| Mean (SD) | 10.08 (20.848) | 4.96 (14.993) | 6.19 (16.707) | 13.30 (28.301) | 21.32 (44.461) |
| Median | 0.90 | 0.30 | 1.25 | 1.30 | 1.60 |
| PT P-value | 0.1220 | 0.2979 | 0.2714 | 0.1714 | 0.1882 |
| PT 95% CI | −3.16, 23.33 | −5.11, 15.04 | −5.76, 18.14 | −6.95, 33.55 | −12.90, 55.50 |
| SW P-value | 0.0004 | 0.0010 | 0.0005 | 0.0005 | 0.0001 |
| WS P-value | 0.2334 | 0.9658 | 0.2324 | 0.2754 | 0.0117 |

ACR = albumin/creatinine ratio;
CI = confidence interval;
EP = Extension Period;
PT = paired t-test;
SD = standard deviation;
SW = Shapiro-Wilk test;
WS = Wilcoxon signed-rank test.

ACR was variable at baseline, ranging from 0.7 to 44.5 ng/L (Table 14), with 2 subjects (Subjects 826-001-007 and 826-001-013) having more advanced disease (>33.9 mg/mmol). Of note, Subject 826-001-007 had an ongoing medical history of microalbuminuria, and Subject 826-001-013 had an ongoing medical history of hypertension. Of note, the largest increases in ACR from baseline occurred in Subjects 826-001-003 (13.8 mg/mmol), 826-001-007 (133.8 mg/mmol), and 826-001-013 (43.9 mg/mmol), each of whom had a break in study drug treatment (32, 32, and 30 days, respectively) between Week 24 and the start of the EP. This suggests that continuous treatment with sodium 2-(3-pentylphenyl)acetate may limit the progression of kidney damage/injury in the subjects.

TABLE 14
Effect of 800 mg sodium 2-(3-pentylphenypacetate on
ACR - individual results

| | ACR (mg/mmol) ^{a} | | | | | |
|---|---|---|---|---|---|---|
| | | | | EP | EP | EP |
| | | | | Week | Week | Week |
| | | | | 12 | 24 | 36 |
| | | Week | Week | (Week | (Week | (Week |
| Subject ID | Baseline | 12 | 24 | 36) | 48) | 60) |
| 826-001-001 ^{b} | 6.3 | 4.3 | 4.2 | 7.5 | 6.2 | 6.1 |
| 826-001-002 | 17.6 | 22.5 | 15.5 | — | — | — |
| 826-001-003^{c,d} | 13.7 | 24.3 | 14.5 | 18.0 | 21.6 | 21.9 |
| 826-001-004^{e} | 17.7 | 14.5 | 10.7 | 7.9 | 6.7 | 19.2 |
| 826-001-005^{f,g} | 0.7 | 0.4 | — | 2.0 | 2.1 | <2.3 |
| 826-001-006^{g} | 4.4 | <2.3 | 2.2 | 2.2 | 0.8 | — |
| 826-001-007^{c} | 44.5 | 89.2 | 77.0 | 95.0 | 111.7 | 178.3 |
| 826-001-009 | 1.7 | <2.3 | <2.3 | <2.3 | 0.7 | 1.9 |
| 826-001-012^{h} | 6.9 | 15.1 | 13.2 | 8.6 | 12.9 | 8.6 |
| 826-001-013^{e} | 41.5 | 102.9 | 77.4 | 56.3 | 106.5 | 85.4 |
| 826-001-014^{d,h} | 18.6 | 15.6 | 10.2 | — | — | — |
| 826-001-015^{h} | 1.1 | <2.3 | 1.4 | 0.6 | <2.3 | <2.3 |

— = no data available; ACR = albumin/creatinine ratio; EP = Extension Period; ID = identification number.
^{a} ACR < 3.39 mg/mmol = normal; ACR 3.39 to 33.9 mg/mmol = early kidney disease (microalbuminuria); and ACR > 33.9 mg/mmol = more advanced kidney disease (macroalbuminuria).
^{b} 204-day break in study drug treatment between Week 24 and EP Day 0 (first day of EP study drug treatment).
^{c}32-day break in study drug treatment between Week 24 and EP Day 0 (first day of EP study drug treatment).
^{d}Insulin-dependent T2D subject.
^{e}30-day break in study drug treatment between Week 24 and EP Day 0 (first day of EP study drug treatment).
^{f}Non-diabetic.
^{g}39-day break in study drug treatment between Week 24 and EP Day 0 (first day of EP study drug treatment).
^{h}Subject had ongoing medical history of chronic kidney disease.

B) Biomarkers of Kidney Injury

Urinary proteins including monocyte chemoattractant protein 1 (MCP-1), kidney injury molecule-1 (KIM-1), clusterin, cystatin C, and osteopontin have been shown to be indicators of kidney injury. The levels of these markers was assessed at baseline and after 24 weeks of treatment with sodium 2-(3-pentylphenyl)acetate in ALMS patients, and the results are reported in Table 15.

TABLE 15
Effect of 800 mg sodium 2-(3-pentylphenyl)acetate on Urinary
Kidney Toxicity Biomarkers

| Urinary | Mean Fold | | | |
|---|---|---|---|---|
| Kidney | Change from | | | |
| Toxicity | Baseline to | | P-Value | P-Value |
| Biomarkers^{a} | Week 24 (%) | SD | (One-tailed) | (Two-tailed) |
| MCP-1 | 0.85 (−15%) | 0.59 | 0.02 | 0.05 |
| Clusterin | 0.66 (−34%) | 0.43 | 0.03 | 0.06 |
| KIM-1 | 0.67 (−33%) | 0.33 | 0.02 | 0.03 |
| Cystatin C | 0.75 (−25%) | 0.29 | 0.01 | 0.02 |
| Osteopontin | 0.82 (−18%) | 0.34 | 0.03 | 0.06 |

KIM-1 = kidney injury marker-1;
MCP-1 = monocyte chemoattractant protein-1;
SD = standard deviation.
^{a}The kidney toxicity panel tested the level of biomarkers in urine samples; subject results were generated as the ratio of the given analyte/creatinine level (ng/nmol).

### Example 5: Effect of 800 mg Sodium 2-(3-Pentylphenyl)Acetate on Metabolic Parameters

ALMS is associated with several metabolic abnormalities. Severe insulin resistance, hyperinsulinemia, and impaired glucose tolerance often present in very early childhood and are frequently accompanied by acanthosis nigricans. T2DM develops in childhood, adolescence, or adulthood, with a mean age of onset at 16 years. Children with ALMS also often have high lipid levels at an early age.

A) Glycated Hemoglobin (HbA1c)

Glycated hemoglobin (HbA1c) is measured primarily to determine the three-month average blood sugar level, and is used as a diagnostic test for diabetes mellitus and as an assessment test for glycemic control in people with diabetes. HbA1c was assessed in ALMS patients treated with sodium 2-(3-pentylphenyl)acetate. There was a statistically significant mean decrease of 0.53% (p=0.0144) in HbA1c from baseline after 12 weeks of treatment (Table 16).

TABLE 161
Effect of 800 mg sodium 2-(3-pentylphenyl)acetate on the Change
from Baseline in Glycated Hemoglobin

| | Change from Baseline HbA1c (%) | | | | | |
|---|---|---|---|---|---|---|
| | | | EP | EP | EP | EP |
| | | | Week 12 | Week 24 | Week 36 | Week 48 |
| Statistics | Week 12 | Week 24 | (Week 36) | (Week 48) | (Week 60) | (Week 72) |
| N | 12 | 12 | 10 | 10 | 9 | 3 |
| Mean (SD) | −0.53 | −0.24 | 0.24 | −0.28 | −0.18 | 0.13 |
| | (0.627) | (0.834) | (1.880) | (1.417) | (0.998) | (0.153) |
| Median | −0.55 | −0.05 | 0.00 | −0.30 | −0.10 | 0.10 |
| PT P-value | 0.0144 | 0.3370 | 0.6958 | 0.5476 | 0.6078 | 0.2697 |
| PT 95% CI | −0.92, | −0.77, | −1.10, | −1.29, | −0.95, | −0.25, |
| | −0.13 | 0.29 | 1.58 | 0.73 | 0.59 | 0.51 |
| SW P-value | 0.3785 | 0.7726 | 0.1014 | 0.0141 | 0.7258 | 0.6369 |
| WS P-value | 0.0225 | 0.4746 | 1.0000 | 0.0781 | 0.7148 | 0.5000 |

CI = confidence interval; HbA1c = glycated hemoglobin; EP = Extension Period; PT = paired t-test; SD = standard deviation; SW = Shapiro-Wilk test; WS = Wilcoxon signed-rank test.

HbA1c was variable at baseline, ranging from 4.8% to 11.9% (Table 17). The lowest baseline HbA1c occurred in Subject 826-001-005 who did not have T2D, and the highest baseline

HbA1c occurred in Subjects 826-001-003 and 826-001-014 who had insulin-dependent T2D. HbA1c decreased or did not change from baseline in the majority of subjects (8/12, 66.7%) after treatment. Of note, Subjects 826-001-007, 826-001-009, and 826-001-014 had their HbA1c reduced by 1% below baseline after study drug treatment; for 2 of these subjects (826-001-007 and 826-001-014), baseline HbA1c was >9%. In contrast, Subject 826-001-001, who had a 204-day break in study drug treatment between Week 24 and the start of the EP, had its HbA1c increased by 1% above baseline by the last study visit.

TABLE 17
By-Subject Listing of Glycated Hemoglobin

| | HbA1c (%) ^{a} | | | | | | |
|---|---|---|---|---|---|---|---|
| | | | | EP | EP | EP | EP |
| | | Week | Week | Week 12 | Week 24 | Week 36 | Week 48 |
| Subject ID | Baseline | 12 | 24 | (Week 36) | (Week 48) | (Week 60) | (Week 72) |
| 826-001-001^{b} | 7.4 | 6.5 | 8.6 | 12 | 10.6 | 9.1 | — |
| 826-001-002 | 5.2 | 5.7 | 6.1 | — | — | — | — |
| 826-001-003^{c,d,e} | 10.8 | 10.1 | 11.1 | 12.7 | 10.6 | 11.2 | 11.1 |
| 826-001-004^{f} | 5.0 | 5.3 | 5.4 | 5.1 | 4.9 | 5.1 | 5.0 |
| 826-001-005^{g,h} | 4.8 | 5.0 | 4.8 | 5.4 | 4.7 | 5.0 | 4.9 |
| 826-001-006^{h} | 6.1 | 5.7 | 5.6 | 5.4 | 5.7 | — | — |
| 826-001-007^{c} | 9.2 | 8.2 | 9.1 | 7.5 | 6.8 | 7.5 | — |
| 826-001-009 | 6.6 | 5.5 | 5.4 | 5.4 | 5.4 | 5.4 | — |
| 826-001-012 | 6.9 | 6.7 | 7.0 | 7.3 | 6.5 | 6.8 | — |
| 826-001-013^{f} | 6.5 | 6.1 | 6.2 | 6.4 | 6.3 | 6.4 | — |
| 826-001-014^{d} | 11.9 | 10.6 | 10.0 | — | — | — | — |
| 826-001-015 | 7.7 | 6.4 | 6.6 | 6.2 | 6.7 | 6.8 | — |

— = no data available; EP = Extension Period; HbA1c = glycated hemoglobin; ID = identification number; T1D = type 1 diabetes; T2D = type 2 diabetes.
^{a} Normal without diabetes 5.6%; normal with diabetes < 6.5%.
^{b}204-day break in study drug treatment between Week 24 and EP Day 0 (first day of EP study drug treatment).
^{c}32-day break in study drug treatment between Week 24 and EP Day 0 (first day of EP study drug treatment).
^{d}Insulin-dependent T2D subject.
^{e}Also had T1D.
^{f}30-day break in study drug treatment between Week 24 and EP Day 0 (first day of EP study drug treatment).
^{g}39-day break in study drug treatment between Week 24 and EP Day 0 (first day of EP study drug treatment).
^{h}Non-diabetic.

B) Fasting Plasma Glucose (FPG)

The effect of sodium 2-(3-pentylphenyl)acetate on FPG levels was assessed, and the results are presented in Table 18.

TABLE 18
Effect of 800 mg sodium 2-(3-pentylphenyl)acetate on the
Change from Baseline in Fasting Plasma Glucose

| | Change from Baseline FPG (mmol/L) | | | | | |
|---|---|---|---|---|---|---|
| | | | EP | EP | EP | EP |
| | | | Week 12 | Week 24 | Week 36 | Week 48 |
| Statistics | Week 12 | Week 24 | (Week 36) | (Week 48) | (Week 60) | (Week 72) |
| N | 11 | 12 | 10 | 10 | 9 | 3 |
| Mean (SD) | 0.30 | 0.33 | 0.63 | 0.70 | 1.49 | −0.93 |
| | (4.958) | (3.594) | (5.234) | (4.571) | (5.380) | (2.065) |
| Median | −0.40 | 0.30 | −0.10 | −0.10 | 0.00 | 0.00 |
| PT P-value | 0.8450 | 0.7600 | 0.7123 | 0.6397 | 0.4305 | 0.5157 |
| PT 95% CI | −3.03, | −1.96, | −3.11, | −2.57, | −2.65, | −6.06, |
| | 3.63 | 2.61 | 4.37 | 3.97 | 5.62 | 4.20 |
| SW P-value | 0.0799 | 0.4366 | 0.0755 | 0.2156 | 0.2299 | 0.2318 |
| WS P-value | 0.5303 | 0.7168 | 0.8203 | 0.9336 | 0.6406 | 1.0000 |

CI = confidence interval; EP = Extension Period; FPG = fasting plasma glucose; PT = paired t-test; SD = standard deviation; SW = Shapiro-Wilk test; WS = Wilcoxon signed-rank test.

FPG was variable at baseline, ranging from 4.7 to 20.0 mmol/L (Table 19). The lowest baseline FPG occurred in Subject 826-001-005 who did not have T2D, and the highest baseline FPG occurred in Subjects 826-001-003 and 826-001-014 who had insulin dependent T2D. Of note, the largest increases in FPG from baseline occurred in Subjects 826-001-001 (7.4 mmol/L) and 826-001-007 (7.9 mmol/L), each of whom had breaks in study drug treatment (204 and 32 days, respectively) between Week 24 and the start of the EP.

TABLE 19
By-Subject Listing of Fasting Plasma Glucose

| | FPG (mmol/L) ^{a} | | | | | | |
|---|---|---|---|---|---|---|---|
| | | | | EP | EP | EP | EP |
| | | Week | Week | Week 12 | Week 24 | Week 36 | Week 48 |
| Subject ID | Baseline | 12 | 24 | (Week 36) | (Week 48) | (Week 60) | (Week 72) |
| 826-001-001^{b} | 7.5 | 5.0 | 13.5 | 19.9 | 13.8 | 14.9 | — |
| 826-001-002 | 5.5 | 4.7 | 6.1 | — | — | — | — |
| 826-001-003^{c,d,e} | 15.8 | 22.8 | 12.9 | 18.0 | 23.3 | 22.7 | 12.5 |
| 826-001-004^{f} | 5.0 | 4.6 | 4.9 | 4.8 | 3.8 | 5.0 | 5.5 |
| 826-001-005^{g,h} | 4.7 | 4.3 | 4.7 | 4.3 | 4.7 | 4.6 | 4.7 |
| 826-001-006^{h} | 6.5 | 5.3 | 5.7 | 5.2 | 6.9 | — | — |
| 826-001-007^{c} | 5.9 | 16.9 | 11.1 | 9.1 | 10.2 | 13.8 | — |
| 826-001-009 | 5.8 | 7.0 | 4.8 | 4.4 | 4.8 | 5.3 | — |
| 826-001-012 | 5.9 | 5.5 | 7.2 | 6.5 | 5.7 | 4.7 | — |
| 826-001-013^{f} | 6.6 | — | 7.4 | 6.6 | 6.2 | 8.6 | — |
| 826-001-014^{d} | 20.0 | 17.6 | 22.6 | — | — | — | — |
| 826-001-015 | 14.3 | 6.5 | 6.5 | 5.5 | 5.6 | 5.3 | — |

— = no data available; EP = Extension Period FPG = fasting plasma glucose; ID = identification number; T1D = type 1 diabetes; T2D = type 2 diabetes.
^{a} Normal FPG
^{b}204-day break in study drug treatment between Week 24 and EP Day 0 (first day of EP study drug treatment).
^{c}32-day break in study drug treatment between Week 24 and EP Day 0 (first day of EP study drug treatment).
^{d}Insulin-dependent T2D subject.
^{e}Also had T1D.
^{f}30-day break in study drug treatment between Week 24 and EP Day 0 (first day of EP study drug treatment).
^{g}39-day break in study drug treatment between Week 24 and EP Day 0 (first day of EP study drug treatment).
^{h}Non-diabetic.

C) Fasting Insulin

The effect of sodium 2-(3-pentylphenyl)acetate on fasting insulin levels was assessed, and the results are presented in Table 20.

TABLE 20
Effect of 800 mg 2-(3-pentylphenyl)acetate on the Change
from Baseline in Fasting Insulin

| | Change from Baseline Fasting Insulin (pmol/L) | | | | | |
|---|---|---|---|---|---|---|
| | | | EP | EP | EP | EP |
| | | | Week 12 | Week 24 | Week 36 | Week 48 |
| Statistics | Week 12 | Week 24 | (Week 36) | (Week 48) | (Week 60) | (Week 72) |
| N | 12 | 12 | 10 | 9 | 8 | 3 |
| Mean (SD) | −18.92 | −339.08 | −920.20 | −1371.78 | −1191.63 | −3933.00 |
| | (1668.350) | (1120.347) | (2685.081) | (4128.701) | (4654.572) | (7237.565) |
| Median | 9.00 | −86.00 | −53.50 | −24.00 | 7.00 | 193.00 |
| PT P-value | 0.9694 | 0.3169 | 0.3067 | 0.3481 | 0.4925 | 0.4459 |
| PT 95% CI | −1078.94, | −1050.92, | −2840.99, | −4545.38, | −5082.94, | −21912.10, |
| | 1041.10 | 372.75 | 1000.59 | 1801.82 | 2699.69 | 14046.11 |
| SW P-value | 0.0022 | 0.0001 | 0.0001 | 0.0001 | 0.0001 | 0.0139 |
| WS P-value | 0.9658 | 0.4238 | 0.2324 | 0.5703 | 0.8438 | 1.0000 |

CI = confidence interval; EP = Extension Period; PT = paired t-test; SD = standard deviation; SW = Shapiro-Wilk test; WS = Wilcoxon signed-rank test.

Fasting insulin was variable at baseline, ranging from 110 to 12,750 μmol/L (Table 21). All but 1 non-diabetic subject (825-001-005) had fasting insulin levels indicative of insulin resistance, with the largest baseline level occurring in an insulin-dependent subject (826-001-003) with both T1D/T2D. Of note, Subject 826-001-003 had a baseline fasting insulin of 12,750 μmol/L that decreased by nearly 30% after 24 weeks and by >96% after 72 weeks of study drug treatment; this subject had insulin dependent T2D. The largest increases in fasting insulin from baseline occurred in Subjects 826-001-001 (420 μmol/L), 826-001-004 (298 μmol/L), 826-001-007 (1,970 μmol/L), and 826-001-013 (1,160 μmol/L), each of whom had a break in treatment (204, 30, 32, and 30 days, respectively) between Week 24 and the start of the EP.

TABLE 21
By-Subject Listing of Fasting Insulin

| | Fasting Insulin (pmol/L) ^{a} | | | | | | |
|---|---|---|---|---|---|---|---|
| | | | | EP | EP | EP | EP |
| | | Week | Week | Week 12 | Week 24 | Week 36 | Week 48 |
| Subject ID | Baseline | 12 | 24 | (Week 36) | (Week 48) | (Week 60) | (Week 72) |
| 826-001-001^{b} | 485 | 105 | 414 | 425 | 880 | 905 | — |
| 826-001-002 | 406 | 467 | 265 | — | — | — | — |
| 826-001-003^{c, d, e} | 12750 | 8400 | 8900 | 4200 | 381 | 202 | 460 |
| 826-001-004^{f} | 302 | 320 | 469 | 370 | 266 | — | 600 |
| 826-001-005^{g, h} | 110 | 135 | 66 | 71 | 92 | 181 | 303 |
| 826-001-006^{h} | 459 | 346 | 358 | 412 | 435 | — | — |
| 826-001-007^{c} | 230 | 2190 | 450 | 330 | 2200 | — | — |
| 826-001-009 | 595 | 3360 | 397 | 305 | 322 | 300 | — |
| 826-001-012 | 315 | 315 | 670 | 441 | 464 | 258 | — |
| 826-001-013^{f} | 420 | 396 | 225 | 157 | 483 | 1580 | — |
| 826-001-014^{d} | 215 | 247 | 222 | — | — | — | — |
| 826-001-015 | 292 | 71 | 74 | 45 | 59 | 38 | — |

— = no data available; EP = Extension Period; ID = identification number; T1D = type 1 diabetes; T2D = type 2 diabetes.
^{a} A fasting insulin level > 174 pmol/L is considered insulin resistance.
^{b}204-day break in study drug treatment between Week 24 and EP Day 0 (first day of EP study drug treatment).
^{c}32-day break in study drug treatment between Week 24 and EP Day 0 (first day of EP study drug treatment).
^{d}Insulin-dependent T2D subject.
^{e}Also had T1D.
^{f}30-day break in study drug treatment between Week 24 and EP Day 0 (first day of EP study drug treatment).
^{g}39-day break in study drug treatment between Week 24 and EP Day 0 (first day of EP study drug treatment).
^{h}Non-diabetic.

D) C-Peptide

The effect of sodium 2-(3-pentylphenyl)acetate on C-peptide levels was assessed, and the results are presented in Table 22.

TABLE 22
Effect of 800 mg 2-(3-pentylphenyl)acetate on the Change
from Baseline in C-peptide

| | Change from Baseline C-Peptide (pmol/L) | | | | | |
|---|---|---|---|---|---|---|
| | | | EP | EP | EP | EP |
| | | | Week 12 | Week 24 | Week 36 | Week 48 |
| Statistics | Week 12 | Week 24 | (Week 36) | (Week 48) | (Week 60) | (Week 72) |
| N | 12 | 12 | 10 | 9 | 8 | 3 |
| Mean (SD) | 473.50 | 41.25 | −350.90 | −286.67 | 219.88 | 810.00 |
| | (2122.903) | (1083.324) | (1144.884) | (891.898) | (1547.287) | (912.692) |
| Median | 102.50 | 84.50 | 6.00 | −40.00 | −31.00 | 737.00 |
| PT P-value | 0.4560 | 0.8974 | 0.3578 | 0.3632 | 0.6997 | 0.2641 |
| PT 95% CI | −875.0, | −647.0, | −1170.0, | −972.0, | −1074.0, | −1457.0, |
| | 1822.0 | 729.6 | 468.1 | 398.9 | 1513.0 | 3077.0 |
| SW P-value | 0.0081 | 0.1961 | 0.3750 | 0.5383 | 0.1249 | 0.8676 |
| WS P-value | 0.3804 | 0.7334 | 0.6250 | 0.5703 | 1.0000 | 0.5000 |

CI = confidence interval; EP = Extension Period; PT = paired t-test; SD = standard deviation; SW = Shapiro-Wilk test; WS = Wilcoxon signed-rank test.

C-peptide was variable at baseline, ranging from 94 to 3,895 μmol/L (Table 23). Most subjects had baseline C-peptide levels outside the normal range. The lowest baseline C-peptide occurred in the 2 insulin-dependent T2D subjects (826-001-003 and 826-001-014), including the 1 subject (826-001-003) with T1D. Of note, low C-peptide levels had been reported to be a biomarker for characterizing at-risk subjects with T1D. Decreases in C-peptide from baseline ranged from 64 to 1,713 μmol/L. Of note, the largest increases in C-peptide from baseline occurred in Subjects 826-001-001 (817 μmol/L), 826-001-004 (1.757 μmol/L), 826-001-005 (737 μmol/L), and 826-001-007 (3,578 μmol/L), each of whom had a break in study drug treatment (204, 30, 39, and 32 days, respectively) between Week 24 and the start of the EP.

TABLE 23
By-Subject Listing of C-Peptide

| | C-Peptide (pmol/L) | | | | | | |
|---|---|---|---|---|---|---|---|
| | | | | EP | EP | EP | EP |
| | | Week | Week | Week 12 | Week 24 | Week 36 | Week 48 |
| Subject ID | Baseline | 12 | 24 | (Week 36) | (Week 48) | (Week 60) | (Week 72) |
| 826-001-001^{b} | 2143 | 988 | 1771 | 2138 | 2949 | 2960 | — |
| 826-001-002 | 1465 | 1807 | 2080 | — | — | — | — |
| 826-001-003^{c, d, e} | 178 | 227 | 139 | 195 | 222 | 282 | 114 |
| 826-001-004^{f} | 2199 | 2639 | 3332 | 3618 | 2589 | — | 3956 |
| 826-001-005^{g, h} | 846 | 876 | 743 | 864 | 806 | 1035 | 1583 |
| 826-001-006^{h} | 1644 | 1850 | 1939 | 1862 | 2284 | — | — |
| 826-001-007^{c} | 1907 | 6065 | 3342 | 2330 | 5485 | — | — |
| 826-001-009 | 2432 | 7570 | 2570 | 1925 | 2077 | 2148 | — |
| 826-001-012 | 3521 | 3677 | 4735 | 2859 | 2348 | 2755 | — |
| 826-001-013^{f} | 3895 | — | 1798 | 1306 | 2769 | 3729 | — |
| 826-001-014^{d} | 94 | 116 | 125 | — | — | — | — |
| 826-001-015 | 2820 | 942 | 1065 | 979 | 1054 | 1107 | — |

— = no data available; EP = Extension Period; ID = identification number; T1D = type 1 diabetes; T2D = type 2 diabetes.
^{a}C-peptide normal range: 364.1-1456.4 pmol/L.
^{b}204-day break in study drug treatment between Week 24 and EP Day 0 (first day of EP study drug treatment).
^{c}32-day break in study drug treatment between Week 24 and EP Day 0 (first day of EP study drug treatment).
^{d}Insulin-dependent T2D subject.
^{e}Also had T1D.
^{f}30-day break in study drug treatment between Week 24 and EP Day 0 (first day of EP study drug treatment).
^{g}39-day break in study drug treatment between Week 24 and EP Day 0 (first day of EP study drug treatment).
^{h}Non-diabetic.

E) Homeostasis Model Assessment for Steady State Beta-Cell Function Based on C-Peptide and Fasting Plasma Glucose (HOMA-B [C-Peptide/FPG])

The effect of sodium 2-(3-pentylphenyl)acetate on HOMA-B (C-peptide/FPG) was assessed, and the results are presented in Table 24.

TABLE 24
Effect of 800 mg 2-(3-pentylphenyl)acetate on the Change from Baseline in HOMA-
B Based on C-Peptide and Fasting Plasma Glucose

| | Change from Baseline HOMA-B Based on C-Peptide and FPG | | | | | |
|---|---|---|---|---|---|---|
| | | | EP | EP | EP | EP |
| | | | Week 12 | Week 24 | Week 36 | Week 48 |
| Statistics | Week 12 | Week 24 | (Week 36) | (Week 48) | (Week 60) | (Week 72) |
| N | 11 | 12 | 10 | 9 | 8 | 3 |
| Mean (SD) | 52.58 | −1.60 | 1.70 | 24.57 | 1.42 | 60.17 |
| | (65.820) | (78.459) | (102.620) | (96.750) | (62.993) | (51.676) |
| Median | 35.60 | 0.60 | 16.50 | −1.90 | 8.00 | 89.40 |
| PT P-value | 0.0243 | 0.9450 | 0.9594 | 0.4681 | 0.9508 | 0.1813 |
| PT 95% CI | 8.36, | −51.45, | −71.71, | −49.80, | −51.24, | −68.20, |
| | 96.80 | 48.25 | 75.11 | 98.94 | 54.09 | 188.54 |
| SW P-value | 0.2817 | 0.8770 | 0.6708 | 0.0395 | 0.8386 | 0.0222 |
| WS P-value | 0.0186 | 0.9697 | 1.0000 | 0.8203 | 1.0000 | 0.2500 |

CI = confidence interval; EP = Extension Period; FPG = fasting plasma glucose; HOMA-B = homeostasis model assessment for steady state beta-cell function; PT = paired t-test; SD = standard deviation; SW = Shapiro-Wilk test; WS = Wilcoxon signed-rank test.

HOMA-B (C-peptide/FPG) was variable at baseline, ranging from 2.4 to 306.4 (Table 25), with the lowest baseline HOMA-B (C-peptide/FPG) occurring in the 2 insulin-dependent T2D subjects (826-001-003 and 826-001-014). HOMA-B (C-peptide/FPG) increased from baseline in most subjects (9/12, 75.0%) after treatment. Of note, the largest reductions in HOMA-B (C-peptide/FPG) occurred in Subjects 826-001-001 (62.4), 826-001-007 (33.4), and 826-001-013 (96.7), each of whom had a break in study drug treatment (204, 32, and 30 days, respectively) between Week 24 and the start of the EP.

TABLE 25
By-Subject Listing of HOMA-B Based on C-Peptide and Fasting Plasma Glucose

| | HOMA-B Based on C-Peptide and FPG | | | | | | |
|---|---|---|---|---|---|---|---|
| | | | | EP | EP | EP | EP |
| | | Week | Week | Week 12 | Week 24 | Week 36 | Week 48 |
| Subject ID | Baseline | 12 | 24 | (Week 36) | (Week 48) | (Week 60) | (Week 72) |
| 826-001-001 ^{a} | 139.7 | 162.8 | 51.3 | 43.8 | 82.5 | 77.3 | — |
| 826-001-002 | 180.3 | 280.3 | 193.8 | — | — | — | — |
| 826-001-003^{b, c, d} | 5.8 | 4.1 | 6.9 | 5.1 | 3.9 | 5.0 | 6.3 |
| 826-001-004^{e} | 288.0 | 382.8 | 405.1 | 446.7 | 538.6 | — | 377.4 |
| 826-001-005^{f, g} | 164.5 | 200.1 | 150.4 | 198.2 | 159.1 | 197.2 | 255.1 |
| 826-001-006^{g} | 145.4 | 228.5 | 207.4 | 237.6 | 168.6 | — | — |
| 826-001-007^{b} | 192.6 | 159.4 | 117.4 | 110.7 | — | 159.2 | — |
| 826-001-009 | 238.0 | 442.9 | 347.4 | 331.6 | 297.8 | 254.8 | — |
| 826-001-012 | 306.4 | 356.8 | 284.1 | 221.8 | 239.0 | 379.7 | — |
| 826-001-013^{e} | 276.3 | — | 124.6 | 119.2 | 234.2 | 179.6 | — |
| 826-001-014^{c} | 2.4 | 3.5 | 2.5 | — | — | — | — |
| 826-001-015 | 76.2 | 96.5 | 105.5 | 135.2 | 137.7 | 158.1 | — |

— = no data available; EP = Extension Period; FPG = fasting plasma glucose; HOMA-B = homeostasis model assessment for steady state beta-cell function; ID = identification number; T1D = type 1 diabetes; T2D = type 2 diabetes.
^{a} 204-day break in study drug treatment between Week 24 and EP Day 0 (first day of EP study drug treatment).
^{b}32-day break in study drug treatment between Week 24 and EP Day 0 (first day of EP study drug treatment).
^{c}Insulin-dependent T2D subject.
^{d}Also had T1D.
^{e}30-day break in study drug treatment between Week 24 and EP Day 0 (first day of EP study drug treatment).
^{f}39-day break in study drug treatment between Week 24 and EP Day 0 (first day of EP study drug treatment).
^{g}Non-diabetic.

### Example 6: Effect of 800 mg Sodium 2-(3-Pentylphenyl)Acetate on Adipose Tissue

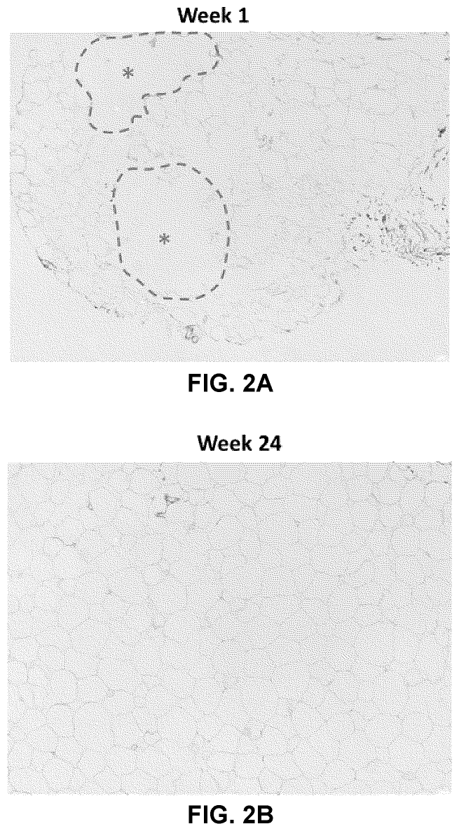

Histological examination of adipocyte tissue in an ALMS subject (before study drug treatment) was characterized by hypertrophy and coalescence of adipocytes, forming giant vesicular vacuolation/steatosis (FIG. 2A). After 24 weeks of treatment with 800 mg 2-(3-pentylphenyl)acetate, more distinct adipocytes were seen that were smaller in size with no observed coalescence (FIG. 2B).

Average adipocyte area decreased from baseline in the majority of subjects (6/9, 66.7%) after 24 weeks of treatment (Table 26). Decreases in average adipocyte area from baseline ranged from 11% to 26%. Of note, the largest increase in average adipocyte area from baseline occurred in Subject 826-001-012 (25%), who had ongoing medical histories of hypothyroidism, myocardial ischemia, T2D, chronic kidney disease, and dyslipidemia. This subject was also characterized as obese with a baseline BMI of 38.13 kg/m².

TABLE 26
Effect of 800 mg sodium 2-(3-pentylphenyl)acetate on
Average Adipocyte Area

| | Average Adipocyte Area (μm²) | | |
|---|---|---|---|
| | | | % Change |
| Subject ID | Baseline | Week 24 | from Baseline |
| 826-001-001 ^{a, b} | NS | 6017.1 | — |
| 826-001-002 ^{b} | 5836.4 | 5898.3 | +1% |
| 826-001-003 ^{b, c, d} | 7286.3 | 6133.7 | −16% |
| 826-001-004 ^{b, e} | 5696.8 | 4197.7 | −26% |
| 826-001-005 ^{f, g} | 4997.4 | 5195.1 | +4% |
| 826-001-006 ^{g} | 5867.0 | 5178.6 | −12% |
| 826-001-007 ^{b, c} | 5599.6 | 4319.0 | −23% |
| 826-001-009 ^{b} | 6790.6 | 6059.9 | −11% |
| 826-001-012 ^{b} | 6547.5 | 8182.0 | +25% |
| 826-001-013 ^{b, e} | 5962.1 | 4792.1 | −20% |
| 826-001-014 ^{b, d} | 4519.7 | NS | — |
| 826-001-015 ^{b} | NS | 4197.3 | — |
| Mean | 6064.9 | 5550.7 | −9% |
| Median | 5867.0 | 5195.1 | −12% |

— = no data available;
ID = identification number;
NS = no sample;
T2D = type 2 diabetes.
^{a} 204-day break in study drug treatment between Week 24 and EP Day 0 (first day of EP study drug treatment).
^{b} Subject had ongoing T2D and/or other metabolic disorders.
^{c} 32-day break in study drug treatment between Week 24 and EP Day 0 (first day of EP study drug treatment).
^{d} Insulin-dependent T2D subject.
^{e} 30-day break in study drug treatment between Week 24 and EP Day 0 (first day of EP study drug treatment).
^{f} Non-diabetic.
^{g} 39-day break in study drug treatment between Week 24 and EP Day 0 (first day of EP study drug treatment).

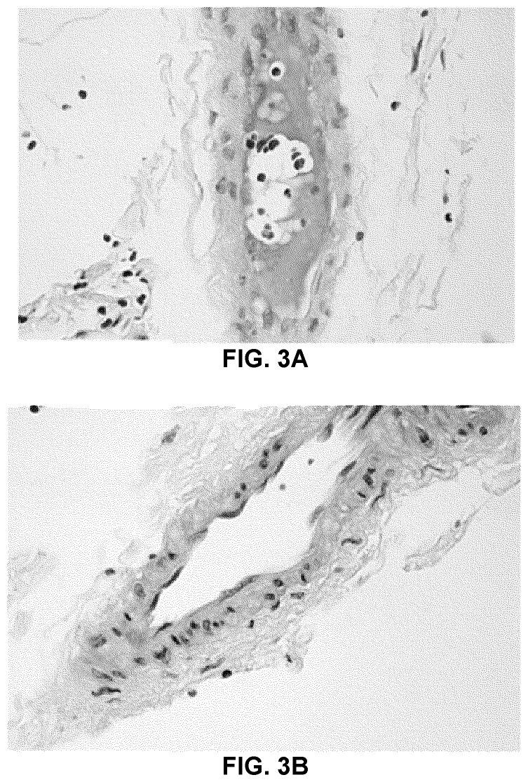

Baseline histological examination of an adipocyte tissue artery (perivascular adipose tissue) in an ALMS subject was characterized by signs of dystrophy, homogenized blurred cytoplasm in smooth muscle cells, and nuclei with irregular shape (FIG. 3A). After 24 weeks of study drug treatment with 800 mg sodium 2-(3-pentylphenyl)acetate, the adipocyte tissue artery looked more normal (FIG. 3B).

The scope of the claims should not be limited by the embodiments set forth in the examples but should be given the broadest interpretation consistent with the description as a whole.

1. A method for improving at least one of liver histology, heart histology, kidney histology, adipose tissue histology and glycemic control in a patient suffering from Alström syndrome (ALMS), said method comprising administering to said patient a pharmaceutical oral formulation comprising 2-(3-pentylphenyl)acetate or a pharmaceutically salt thereof and a pharmaceutically acceptable excipient, wherein said 2-(3-pentylphenyl)acetate or a pharmaceutically salt thereof is administered at a daily dose of about 500 mg to about 1500 mg for a treatment period of more than 48 weeks, and wherein the administration is not interrupted for 15 days or more during said treatment period.

2. The method of claim 1, wherein the treatment period is of more than 54 weeks.

3. (canceled)

4. The method of claim 1, wherein the administration is not interrupted for 30 days or more.

5. The method of claim 1, wherein said method is for improving at least liver histology.

6. The method of claim 5, wherein improving liver histology comprises reducing liver stiffness and/or reducing the Metavir score by at least one grade or stage.

7. The method of claim 6, wherein the liver stiffness is reduced by at least 2 kPa relative to prior to the administration, as measured by transient elastography.

8. (canceled)

9. (canceled)

10. The method of claim 1, wherein said method is for improving at least heart histology.

11. The method of claim 1, wherein improving heart histology comprises increasing left ventricular function (LVF) and/or reducing short-axis (plane) longitudinal relaxation time (SAX T1).

12. The method of claim 11, wherein the LVF comprises left ventricular end-diastolic volume (LVEDV) and/or left ventricular end-systolic volume (LVESV).

13. (canceled)

14. The method of claim 12, wherein the method is for increasing LVEDV by at least 6 ml relative to prior to the administration, as measured by cardiac Magnetic Resonance Imaging (MRI).

15. (canceled)

16. (canceled)

17. The method of claim 11, wherein the method is for reducing Basal SAX T1 by at least 40 ms and/or Mid SAX T1 by at least 25 ms relative to prior to the administration, as measured by cardiac MRI.

18. (canceled)

19. The method of claim 1, wherein said method is for improving at least kidney histology.

20. (canceled)

21. The method of claim 19, wherein improving kidney histology comprises reducing the levels of one or more biomarkers of kidney injury selected from monocyte chemoattractant protein 1 (MCP-1), kidney injury molecule-1 (KIM-1), clusterin, cystatin C and osteopontin by at least 10% relative to prior to the administration.

22. (canceled)

23. (canceled)

24. The method of claim 1, wherein said method is for improving at least adipose tissue histology.

25. The method of claim 24, wherein improving adipose tissue histology comprises reducing average adipocyte area by at least 10%.

26. (canceled)

27. The method of claim 1, wherein said method is for improving at least glycemic control.

28. The method of claim 27, wherein improving glycemic control comprises reducing glycated hemoglobin (HbA1c) by at least 1% (absolute) and/or increasing the homeostasis Model Assessment for Steady State Beta-Cell Function Based on C-Peptide and Fasting Plasma Glucose (HOMA-B [C-Peptide/FPG]) by at least 20.

29. The method of claim 28, wherein the subject has an HbA1c of more than 9% prior to administration.

30. (canceled)

31. (canceled)

32. (canceled)

33. (canceled)

34. The method of claim 27, wherein the patient has type 2 diabetes.

35. (canceled)

36. (canceled)

37. The method of claim 6, wherein the patient has fibrosis stage F2, F3 or F4 according to the Metavir scoring system.

38. (canceled)

39. (canceled)

40. (canceled)

41. (canceled)

42. The method of claim 14, wherein (i) the patient is a male and has a LVEDV of less than 106 mL and/or a LVESV of less than 26 mL; or (ii) the patient is a female and has a LVEDV of less than 86 mL and/or a LVESV of less than 22 mL.

43. (canceled)

44. (canceled)

45. (canceled)

46. (canceled)

47. (canceled)

48. (canceled)

49. (canceled)

50. (canceled)

51. The method of claim 1, wherein the daily dose is of about 700 mg to about 900 mg or about 1100 mg to about 1300 mg.

52. (canceled)

53. (canceled)

54. (canceled)

55. The method of claim 1, wherein the pharmaceutical oral formulation comprises sodium 2-(3-pentylphenyl)acetate.

56. (canceled)

57. The method of claim 1, wherein the subject (i) has a body mass index (BMI)≥25.0 kg/m², and/or (ii) is a male and has a waist circumference ≥94 cm, or is a female and has a waist circumference ≥80 cm.

58. (canceled)

59-116. (canceled)
